(19) United States
(12) Patent Application Publication
(10) Pub. No.: US 2008/0183246 A1
(43) Pub. Date: Jul. 31, 2008

(54) Method, apparatus and system for guiding a procedure relating to an implantable medical device

(75) Inventors: Sejal B. Patel, Oklahoma City, OK (US); Jason D. Begnaud, Houston, TX (US); Chris G. DuPont, League City, TX (US); Albert A. Rodriguez, Friendswood, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/698,688

(22) Filed: Jan. 26, 2007

(51) Int. Cl. A61N 1/36

(52) U.S. Cl. 607/60

(57) ABSTRACT

A method, apparatus, and system, are provided for guiding a medical procedure relating to an implantable medical device operatively coupled to a cranial nerve. Communications between the implantable medical device and an external device are established. An implant procedure is performed for implanting the implantable medical device. A first diagnostic process of the implantable medical device is performed. Using the external device a first signal is received from the implantable medical device based on the first diagnostic process. A first instruction is displayed using the external device based upon the first signal received by the external device. The first instruction includes information relating to guiding the implant procedure.

### BACKGROUND OF THE INVENTION

1. Field of the Invention

This invention relates generally to implantable medical device systems and, more particularly, a guided procedure function to an interactive forum for providing a navigated set of displays for implanting an implantable medical device (IMD) and/or performing a patient care management action during a follow-up visit to a physician to adjust parameter data for treating one or more disorders using an IMD.

2. Description of the Related Art

Many advancements have been made in treating diseases such as epilepsy. Therapies using electrical signals for treating these diseases have been found to effective. Implantable medical devices have been effectively used to deliver therapeutic stimulation to various portions of the human body (e.g., the vagus nerve) for treating these diseases. As used herein, “stimulation” or “stimulation signal” refers to the application of an electrical, mechanical, magnetic, electromagnetic, photonic, audio and/or chemical signal to a neural structure in the patient's body. The signal is an exogenous signal that is distinct from the endogenous electrical, mechanical, and chemical activity (e.g., afferent and/or efferent electrical action potentials) generated by the patient's body and environment. In other words, the stimulation signal (whether electrical, mechanical, magnetic, electromagnetic, photonic, audio or chemical in nature) applied to the nerve in the present invention is a signal applied from an artificial source, e.g., a neurostimulator.

A “therapeutic signal” refers to a stimulation signal delivered to a patient's body with the intent of treating a disorder by providing a modulating effect to neural tissue. The effect of a stimulation signal on neuronal activity is termed “modulation”; however, for simplicity, the terms “stimulating” and “modulating”, and variants thereof, are sometimes used interchangeably herein. In general, however, the delivery of an exogenous signal itself refers to “stimulation” of the neural structure, while the effects of that signal, if any, on the electrical activity of the neural structure are properly referred to as “modulation.” The modulating effect of the stimulation signal upon the neural tissue may be excitatory or inhibitory, and may potentiate acute and/or long-term changes in neuronal activity. For example, the “modulating” effect of the stimulation signal to the neural tissue may comprise one more of the following effects: (a) initiation of an action potential (afferent and/or efferent action potentials); (b) inhibition or blocking of the conduction of action potentials, whether endogenous or exogenously induced, including hyperpolarizing and/or collision blocking, (c) affecting changes in neurotransmitter/neuromodulator release or uptake, and (d) changes in neuro-plasticity or neurogenesis of brain tissue.

Electrical neurostimulation may be provided by implanting an electrical device underneath the skin of a patient and delivering an electrical signal to a nerve such as a cranial nerve. In one embodiment, the electrical neurostimulation involves sensing or detecting a body parameter, with the electrical signal being delivered in response to the sensed body parameter. This type of stimulation is generally referred to as “active,” “feedback,” or “triggered” stimulation. In another embodiment, the system may operate without sensing or detecting a body parameter once the patient has been diagnosed with a medical condition that may be treated by neurostimulation. In this case, the system may apply a series of electrical pulses to the nerve (e.g., a cranial nerve such as a vagus nerve) periodically, intermittently, or continuously throughout the day, or over another predetermined time interval. This type of stimulation is generally referred to as “passive,” “non-feedback,” or “prophylactic,” stimulation. The electrical signal may be applied by an IMD that is implanted within the patient's body. In another alternative embodiment, the signal may be generated by an external pulse generator outside the patient's body, coupled by an RF or wireless link to an implanted electrode.

Generally, neurostimulation signals that perform neuromodulation are delivered by the IMD via one or more leads. The leads generally terminate at their distal ends in one or more electrodes, and the electrodes, in turn, are electrically coupled to tissue in the patient's body. For example, a number of electrodes may be attached to various points of a nerve or other tissue inside a human body for delivery of a neurostimulation signal.

During a surgical operation to implant an IMD into a patient's body, various steps are performed to insure proper operation of the IMD throughout the implant surgery. Generally, these steps are performed as part of a manual list of tasks that have been developed to ensure proper functionality of the IMD. In some cases, a trouble-shooting, step-by-step guide may be consulted as part of the manual process of implanting and verifying proper operation of the IMD. When improper operation is detected, a further series of manual steps may be taken to correct any malfunction. One of the problems associated with the state-of-the-art methodology of implanting medical devices is that the series of manual steps may be excessively time consuming. For example, when improper operation of the device is detected during implantation of a medical device, various manual checks may be performed to correct any problems. Because these manual tasks may take considerable time to perform, the surgical implant procedure may require additional operating room time.

When employing state-of-the-art methodology of implanting medical devices using the manual system, some errors may be inadvertently overlooked. For example, some communication errors may not be recognized by a medical professional during the installation process. Further, interpretive errors may exist as a result of the manual process involving state-of-the-art implantation of medical devices. For example, if a communication error is detected, an incorrect step of checking the pin connections between a lead and the IMD may be performed, which may not shed light on the detected problem. Other interpretive errors may also be encountered using the state-of-the-art implantation devices. In addition, the surgeon may inadvertently omit one or more verification or troubleshooting steps in the surgical process.

State-of-the-art implantable medical systems utilize an external device (ED) to communicate with the IMD for programming the therapeutical electrical signal to be delivered by the implanted device, performing diagnostics and making adjustments to one or more parameters defining the therapeutic electrical signal. A physician may assess the progress of a particular therapy regimen given to a patient during office visits following surgical implant. The physician may examine the patient and make a determination as to the efficacy of the therapy being delivered and may use the ED to reprogram or adjust various stimulation parameters that will modify subsequent therapy delivered to the patient.

There are various problems associated with state-of-the-art implanted neurostimulators. For example, tedious record-keeping and study of charts are required to perform therapy management to treat patients. When the physician evaluates a patient, various settings for therapy delivered by the IMD are documented in the patient's chart at each visit. At subsequent visits, the physician may then examine previous entries into the chart (e.g., the physician may study the various parameters defining the therapeutic electrical signal, medications taken by the patient, etc.) to make adjustments to the therapy delivered by the IMD. The process of documenting the changes in the parameters, medication, and patient evaluation may become quite laborious, as well as time-consuming, with a corresponding risk that important information may not be collected or may not be incorporated into the adjustments made to the therapy to improve or maintain efficacy.

Further, during a follow-up visit with a medical professional, access to a patient's IMD may not be performed efficiently due to a lack of knowledge of the IMD's capabilities and/or due to a failure to collect data available from the IMD. State-of-the-art methods involve a medical professional manually checking various performance data relating to the IMD and making certain observations based upon the data thus obtained. However, the medical professional may not be aware of the various recommended steps and tests that may be performed to gain a more in-depth analysis of the performance of the IMD.

The present invention is directed to overcoming, or at least reducing, the effects of one or more of the problems set forth above.

### SUMMARY OF THE INVENTION

In one aspect, the present invention provides a method for guiding a medical procedure relating to an implantable medical device (IMD) operatively coupled to a cranial nerve. Communications between the IMD and an external device (ED) are established. An implant procedure is performed for implanting the IMD. A first diagnostic process of the IMD is performed. Using the ED, a first signal is received from the IMD based on the first diagnostic process. A first instruction is displayed using the ED based upon the first signal received by the ED. The first instruction includes information relating to guiding the implant procedure.

In another aspect, the present invention provides a method for guiding a medical procedure relating to an IMD operatively coupled to a cranial nerve. Communications between the IMD and an ED is established. A diagnostic process is performed on the IMD. The ED receives a first signal from the IMD as a result of the diagnostic process. At least one selectable option is displayed using the ED based upon the signal received by the ED. The selectable option relates to an operational setting of the IMD.

In another aspect, the present invention provides a method for guiding a medical procedure relating to an IMD operatively coupled to a cranial nerve. Communications between the IMD and an ED is established. A first signal is transmitted from the ED to the IMD. Using the ED, a second signal is received from the IMD responsive to the first signal. A recommendation is determined using the ED, in response to the second signal. A display indicative of the recommendation is generated using the ED.

In another aspect, a graphical user interface (GUI) integrated into an ED is provided for guiding an operation relating to an IMD coupled to a cranial nerve. The GUI includes a display region adapted to display a visual indication of a graphical representation, confirming the establishment of communications between the IMD and the ED. The ED transmits a first signal to the IMD. The ED also receives a second signal from the IMD responsive to the first signal. Additionally, the ED determines a recommendation in response to the second signal. The display region also provides a display indicative of the recommendation.

In yet another aspect of the present invention, a system is provided for guiding an operation of an IMD operatively coupled to a cranial nerve. The system of the present invention includes an IMD capable of providing an electrical signal for treating a disease. The system also includes an external computing device (ECD) capable of communicating with the IMD. The ECD is capable of establishing communications with the IMD and transmitting a first signal from the ECD to the IMD. The ECD is also capable of receiving a second signal from the IMD responsive to the first signal, determining a recommendation in response to the second signal, and generating a display indicative of the recommendation using the ECD.

In yet another aspect of the present invention, a computer readable program storage device encoded with instructions is provided for guiding an operation of an IMD operatively coupled to a cranial nerve. The computer readable program storage device is encoded with instructions that, when executed by a computer, performs a method, which comprises: establishing communications between the IMD and an ED; performing a diagnostic process on the IMD; receiving with the ED a first signal from the IMD as a result of the diagnostic process; and displaying at least one selectable option using the ED based upon the signal received by the ED. The selectable option relates to an operational setting of the IMD.

### BRIEF DESCRIPTION OF THE DRAWINGS

The invention may be understood by reference to the following description taken in conjunction with the accompanying drawings, in which like reference numerals identify like elements, and in which:

FIG. 1 provides a stylized diagram of an implantable medical device implanted into a patient's body for providing a therapeutic electrical signal to a neural structure of the patient's body, in accordance with one illustrative embodiment of the present invention;

FIG. 2A is a block diagram of a medical device system that includes an implantable medical device and an external device that includes a graphical user interface unit for providing a patient management system for the implantable medical device, in accordance with one illustrative embodiment of the present invention;

FIG. 2B is a block diagram of a medical device system that includes an implantable medical device and an external device that includes a graphical user interface unit for providing a patient management system for the implantable medical device, in accordance with an alternative illustrative embodiment of the present invention;

FIG. 3 is a more detailed block diagram of the interface unit of FIG. 2B, in accordance with one illustrative embodiment of the present invention;

FIG. 4 is a more detailed block diagram depiction of a procedure guidance unit of FIG. 2A or 2B, in accordance with one illustrative embodiment of the present invention;

FIG. 5 is a flowchart of a method of performing the guided procedure function relating to an IMD, in accordance with one illustrative embodiment of the present invention

FIGS. 6-44 depict various screens (e.g., GUI displays) that may be displayed by the GUI unit of FIGS. 2A and 2B for performing various guided procedure functions relating to an implantable medical device, in accordance with one illustrative embodiment of the present invention.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

This document does not intend to distinguish between components that differ in name but not function. In the following discussion and in the claims, the terms “including” and “includes” are used in an open-ended fashion, and thus should be interpreted to mean “including, but not limited to.” Also, the term “couple” or “couples” is intended to mean either a direct or an indirect electrical connection. “Direct contact,” “direct attachment,” or providing a “direct coupling” indicates that a surface of a first element contacts the surface of a second element with no substantial attenuating medium there between. The presence of substances, such as bodily fluids, that do not substantially attenuate electrical connections does not vitiate direct contact. The word “or” is used in the inclusive sense (i.e., “and/or”) unless a specific use to the contrary is explicitly stated.

The term “electrode” or “electrodes” described herein may refer to one or more stimulation electrodes (i.e., electrodes for delivering an electrical signal generated by an IMD to a tissue), sensing electrodes (i.e., electrodes for sensing a physiological indication of a patient's body), and/or electrodes that are capable of delivering a stimulation signal, as well as performing a sensing function.

Cranial nerve stimulation has been proposed to treat a number of disorders pertaining to or mediated by one or more structures of the nervous system of the body, including epilepsy and other movement disorders, depression, anxiety disorders and other neuropsychiatric disorders, dementia, head trauma, coma, migraine headache, obesity, eating disorders, sleep disorders, cardiac disorders (such as congestive heart failure and atrial fibrillation), hypertension, endocrine disorders (such as diabetes and hypoglycemia), and pain, among others. See, e.g., U.S. Pats. Nos. 4,867,164; 5,299,569; 5,269,303; 5,571,150; 5,215,086; 5,188,104; 5,263,480; 6,587,719; 6,609,025; 5,335,657; 6,622,041; 5,916,239; 5,707,400; 5,231,988; and 5,330,515. Despite the numerous disorders for which cranial nerve stimulation has been proposed or suggested as a treatment option, the fact that detailed neural pathways for many (if not all) cranial nerves remain relatively unknown, makes predictions of efficacy for any given disorder difficult or impossible. Moreover, even if such pathways were known, the precise stimulation parameters that would modulate particular pathways relevant to a particular disorder generally cannot be predicted.

Despite the difficulties of predicting efficacy for particular disorders, the use of vagus nerve stimulation as a therapy for treating epilepsy and/or depression is an established therapy option. Although many patients respond well to the therapy, a significant number of patients require periodic follow-up visits to have the therapeutic electrical signal adjusted periodically to cause and/or maintain a positive therapeutic response. Further, implanting the IMD into a patient's body may be a time-consuming process that involves precise steps for trouble-shooting to ensure proper installation of the IMD. The present invention provides guided procedure functions for guiding a user during a surgical operation for implanting an IMD into a patient's body and/or for performing a guided process during a post-implant follow-up visit to a physician's office to verify or improve the efficacy of the stimulation provided by the IMD. The guided procedure function of the present invention may refer to a series of interactions between an IMD, an ED (e.g., a computer, a hand-held device, etc.) communicatively coupled to the IMD, and a user (e.g., a medical professional). Guided procedure function may refer to various recommendations made to the user, via a display (e.g., a graphical user interface display) that is connected to the ED. The ED may communicate with the IMD, and based upon this communication, the ED may provide a series of recommended or suggested steps/procedures to the user based upon inputs from the IMD and/or the user. The guided procedure function may guide a medical professional through procedures, such as a surgical procedure for implanting a medical device, a follow-up visit to evaluate the efficacy of the therapy and other operating parameters relating to the IMD, and/or implementing changes to the operation of the IMD by adjusting one or more operating parameters.

The implantable medical system of the present invention provides for software module(s) that are capable of acquiring, storing, and processing various forms of data, such as patient data/parameters (e.g., physiological data, side-effects data, such as effects on heart rate and breathing, brain-activity parameters, disease progression or regression data, self-evaluation data, seizure characteristic data, quality of life data, etc.) and therapy parameter data. The guided procedure function of embodiments of the present invention provides for various tasks, such as guiding a surgical implantation procedure, guiding a follow-up evaluation process, guiding the adjustment of various therapy parameters, etc. Therapy parameters may include, but are not limited to, electrical signal parameters that define the therapeutic electrical signals delivered by the IMD, medication parameters (e.g., dosages, frequency of medication provided to the patient, etc.) and/or any other therapeutic treatment parameter. In an alternative embodiment, the term “therapy parameters” may refer to electrical signal parameters defining the therapeutic electrical signals delivered by the IMD. Therapy parameters for a therapeutic electrical signal may also include, but are not limited to, a current amplitude, a voltage amplitude, a rate of change of said current amplitude, a rate of change of said voltage amplitude, a time period of a rate of change of said current amplitude, a time period of a rate of change of said voltage amplitude, a pulse width, a rate of change of the pulse width, a time period of a rate of change of the pulse width, a frequency, a rate of change of the frequency, a time period of a rate of change of the frequency, a signal on-time, a signal off-time, and/or a duty cycle.

In one embodiment, the guided procedure function of the present invention also provides for a plurality of graphical user interfaces (GUI) that may be interactive. The GUIs of the guided procedure function may facilitate the entry of various physicians' inputs, such as the selection of a mode of operation for a guided procedure. Once a certain mode, such as a surgical implant mode, a follow-up visit mode, a user preferences mode, etc., is initiated by a user (e.g., a physician, a nurse, or a medical technician), the guided procedure function may provide, for example, an interactive interface to perform trouble-shooting and installation procedures during a surgical implant process, or to perform adjustments to the operations of the IMD during a follow-up office visit and/or adjust various operation parameters of the IMD.

Embodiments of the present invention provide for a method, apparatus and system for providing communications with an IMD using an ED. The embodiments of the present invention provide for communications between the ED and the IMD to provide guided installation instructions during a pre-operation, operation and/or a post-operation procedure relating to implantation of the IMD. Further, embodiments of the present invention provide for an instructional or navigated set of screens for providing instructions on performing various post-surgical follow-up interactions with the IMD. These interactions may include various tasks, such as inquiry of IMD performance data, programming of various operational parameters, i.e., details regarding the type of stimulation data and the frequency of stimulation delivery, etc.

Embodiments of the present invention provide for a navigated set of instructional screens on an ED that may be useful during pre-operation, operation and post-operation procedures for implanting an IMD in a patient's body. For example, during implant, various problems, such as communications errors between the external device and the IMD, may exist. Various trouble-shooting instructional text, graphics, animation, etc., may be provided via the ED to inform the surgeon how to resolve the various implant problems as they occur. For example, if communication between the ED and the IMD occurs during implantation of the IMD into a patient's body, various trouble-shooting steps may be automatically provided by the ED in embodiments of the present invention. This may include instructions, such as repositioning the hand-held device or the wand (a communication device typically coupled to the ED for placing externally over the patient's skin in close proximity to the implanted IMD, to establish and permit communications between the ED and the IMD) for better communication; changing or charging the batteries associated with the ED; adjusting the depth of the position of the IMD, etc. The term “wand” may in alternative embodiments refer to a hand-held device that is capable of performing the various computational processes described herein. The term “wand” may also refer to a device that is an extension of a data interface device that is coupled operatively to a stand-alone computer device and/or a hand-held device. In embodiments of the present invention, the wand (where present) is considered to be part of the ED system for communicating with the IMD. In one embodiment, the wand may be the entire ED. In an alternative embodiment, the wand may be a portion of an ED.

Various instructions to assist in the step-by-step procedure of implantation of the IMD may be provided by the ED. For example, the ED may instruct a medical professional (e.g., a physician) to perform a lead impedance test. The result of the lead impedance test may be detection of a high lead impedance in the operating room (during the implantation process). The implant procedure may include a pre-operation phase, an operation phase, and a post-operation phase. Various tasks or steps may be suggested by the ED during the implantation process. A high lead impedance during an installation process may be caused by various factors, such as the lead being improperly interfaced with a target nerve in the patient's body, one or more electrodes being improperly interfaced with a target nerve, a lead pin associated with the lead being improperly inserted into the IMD, a break in the lead, etc.

In response to the detection of a high lead impedance during an implant procedure, the ED of the embodiments of the present invention may provide various instructions or recommendations to the surgeon, such as pulling the pin out of the IMD, checking inside the header of the IMD to insure that the set screw associated with the header is not obstructing the lead insertion, and/or other troubleshooting steps. For example, if in response to a recommendation to check the set screw inside the header of the IMD, the surgeon observes that the set screw inside the header of an IMD is, indeed, obstructing the lead insertion, the ED may then provide instructions to back-up the setscrew(s), fully insert the lead and tighten the set screw in the header. In some cases, different or additional instructions such as disconnecting the lead from the IMD and using a test resistor to check the lead impedance may be provided in order to ensure that the IMD itself is not causing such a problem. These various actions and proposed solutions may be automatically provided by the ED through interactive prompts displayed to, and responses provided by, the surgeon. This may reduce interpretive errors and the possibility of overlooking various errors since they may be automatically flagged utilizing embodiments of the present invention. Various methods may be used to convey instructions to a medical professional, such as a series of instructive screens providing text and/or graphics to prompt a sequence of steps to be performed by a user. In this manner, a navigated set of screens may be useful for problem-solving/trouble-shooting during a surgical implant procedure, as well as for a guided follow-up procedure during, e.g., a post-implant office visit.

The guided follow-up procedure may involve a post-operation visit to a medical professional at various pre-determined and/or random intervals to check the operation of the IMD. Various instructional guidance screens may be provided by the external device to prompt a user to perform various tests and/or adjustments of parameters associated with the operation of the IMD. The guided navigated set of screens provided by the embodiments of the present invention for pre-operation, post-operation, and/or follow-up visits are described in further details in various drawings and the accompanying descriptions below.

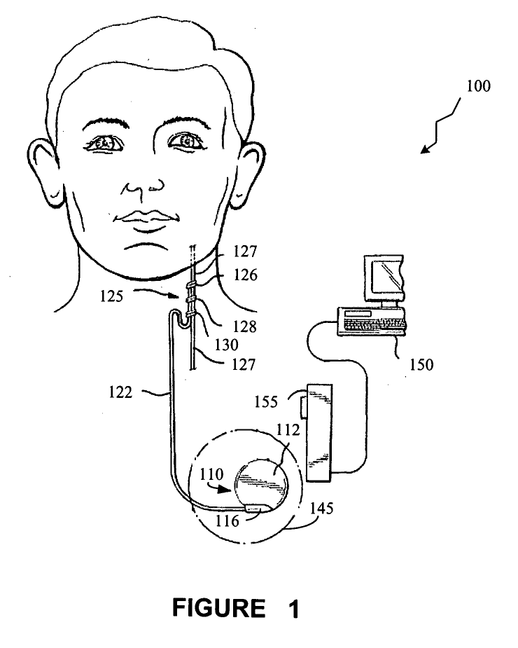

Although not so limited, a system capable of implementing embodiments of the present invention is described below. FIG. 1 depicts a stylized implantable medical system 100 for implementing one or more embodiments of the present invention. An electrical signal generator 110 is provided, having a main body 112 comprising a case or shell with a header 116 for connecting to an insulated, electrically conductive lead assembly 122. The generator 110 is implanted in the patient's chest in a pocket or cavity formed by the implanting surgeon just below the skin (indicated by a dotted line 145, similar to the implantation procedure for a pacemaker pulse generator.

A nerve electrode assembly 125, preferably comprising a plurality of electrodes having at least an electrode pair, is conductively connected to the distal end of the lead assembly 122, which preferably comprises a plurality of lead wires (one wire for each electrode). Each electrode in the electrode assembly 125 may operate independently or alternatively, may operate in conjunction with the other electrodes.

Lead assembly 122 is attached at its proximal end to connectors on the header 116 of generator 110. The electrode assembly 125 may be surgically coupled to a vagus nerve 127 in the patient's neck or at another location, e.g., near the patient's diaphragm or at the esophagus/stomach junction. Other (or additional) cranial nerves such as the trigeminal and/or glossopharyngeal nerves may also be used to deliver the electrical signal in particular alternative embodiments. In one embodiment, the electrode assembly 125 comprises a bipolar stimulating electrode pair 126, 128. Suitable electrode assemblies are available from Cyberonics, Inc., Houston, Tex., USA as the Model 302 electrode assembly. However, persons of skill in the art will appreciate that many electrode designs could be used in the present invention. In one embodiment, the two electrodes are wrapped about the vagus nerve, and the electrode assembly 125 may be secured to the nerve 127 by a spiral anchoring tether 130 such as that disclosed in U.S. Pat. No. 4,979,511 issued Dec. 25, 1990 to Reese S. Terry, Jr. and assigned to the same assignee as the instant application. Lead assembly 122 is secured, while retaining the ability to flex with movement of the chest and neck, by a suture connection to nearby tissue (not shown).

In alternative embodiments, the electrode assembly 125 may comprise temperature sensing elements and/or heart rate sensor elements. Other sensors for other body parameters may also be employed to trigger active stimulation. Both passive and active stimulation may be combined or delivered by a single IMD according to the present invention. Either or both modes may be appropriate to treat a specific patient under observation.

The electrical pulse generator 110 may be programmed with an ED such as computer 150 using programming software based on the description herein. A programming wand 155 may be coupled to the computer 150 as part of the ED to facilitate radio frequency (RF) communication between the computer 150 and the pulse generator 110. The programming wand 155 and computer 150 permit non-invasive communication with the generator 110 after the latter is implanted. In systems where the computer 150 uses one or more channels in the Medical Implant Communications Service (MICS) bandwidths, the programming wand 155 may be omitted to permit more convenient communication directly between the computer 150 and the pulse generator 110.

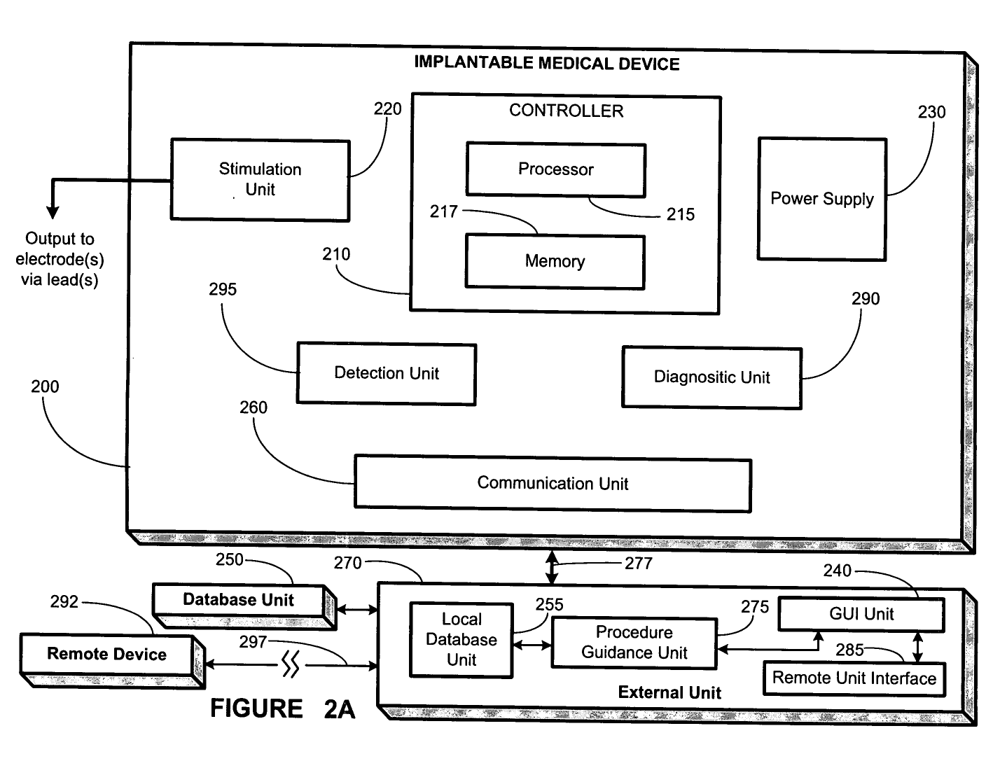

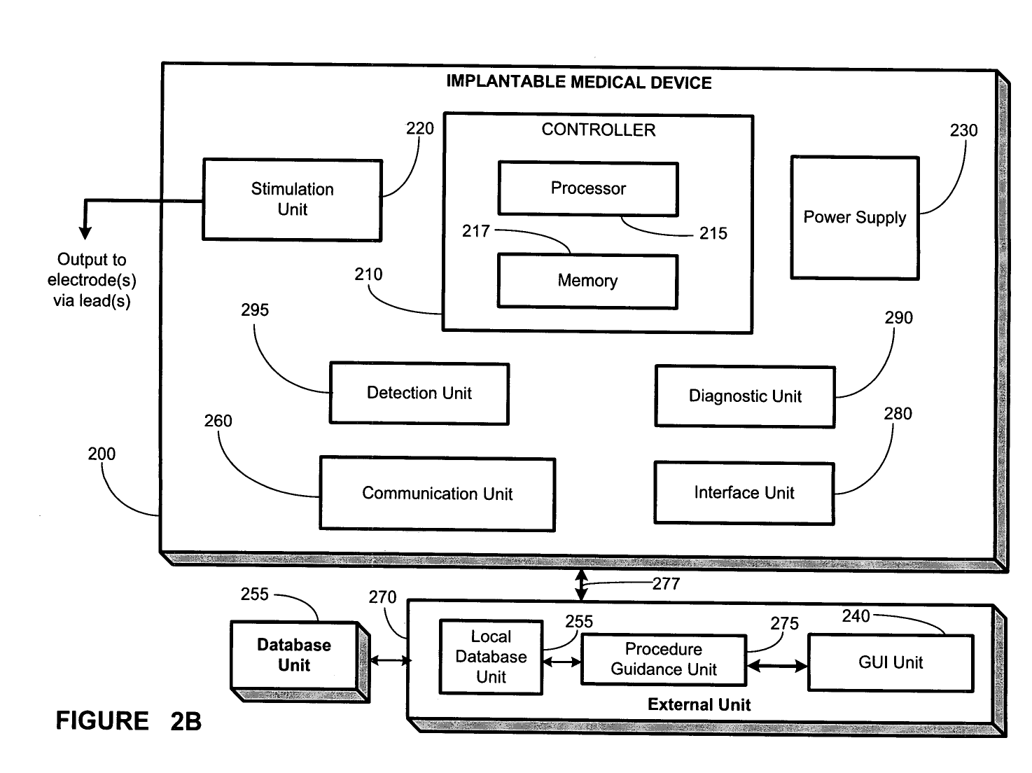

Turning now to FIG. 2A, a block diagram depiction of the IMD 200 is provided, in accordance with one illustrative embodiment of the present invention. The IMD 200 (such as generator 110 from FIG. 1) may comprise a controller 210 capable of controlling various aspects of the operation of the IMD 200. The controller 210 is capable of receiving internal data or external data and causing a stimulation unit 220 to generate and deliver an electrical signal to target tissues of the patient's body for treating a medical condition. For example, the controller 210 may receive manual instructions from an operator externally, or may cause the electrical signal to be generated and delivered based on internal calculations and programming. The controller 210 is capable of affecting substantially all functions of the IMD 200.

The controller 210 may comprise various components, such as a processor 215, a memory 217, etc. The processor 215 may comprise one or more microcontrollers, microprocessors, etc., capable of performing various executions of software components. The memory 217 may comprise various memory portions where a number of types of data (e.g., internal data, external data instructions, software codes, status data, diagnostic data, etc.) may be stored. The memory 217 may comprise one or more of random access memory (RAM) dynamic random access memory (DRAM), electrically erasable programmable read-only memory (EEPROM), flash memory, etc.

The IMD 200 may also comprise a stimulation unit 220 capable of generating and delivering electrical signals to one or more electrodes via leads. A lead assembly such as lead assembly 122 (FIG. 1) may be coupled to the IMD 200. Therapy may be delivered to the leads comprising the lead assembly 122 by the stimulation unit 220 based upon instructions from the controller 210. The stimulation unit 220 may comprise various circuitry, such as stimulation signal generators, impedance control circuitry to control the impedance “seen” by the leads, and other circuitry that receives instructions relating to the delivery of the electrical signal to tissue. The stimulation unit 220 is capable of delivering a controlled current electrical signal over the leads comprising the lead assembly 122.

The IMD 200 may also comprise a power supply 230. The power supply 230 may comprise a battery, voltage regulators, capacitors, etc., to provide power for the operation of the IMD 200, including delivering the therapeutic electrical signal. The power supply 230 comprises a power source that in some embodiments may be rechargeable. In other embodiments, a non-rechargeable power source may be used. The power supply 230 provides power for the operation of the IMD 200, including electronic operations and the electrical signal generation and delivery functions. The power supply 230 may comprise a lithium/thionyl chloride cell or a lithium/carbon monofluoride (LiCFx) cell. Other battery types known in the art of implantable medical devices may also be used.

The IMD 200 may also comprise a communication unit 260 capable of facilitating communications between the IMD 200 and various devices. In particular, the communication unit 260 is capable of providing transmission and reception of electronic signals to and from an external unit 270, such as computer 150 and wand 155 that may comprise an ED (FIG. 1). The communication unit 260 may include hardware, software, firmware, or any combination thereof.

The IMD 200 also comprises a detection unit 295 that is capable of detecting various patient parameters. For example, the detection unit 295 may comprise hardware, software, or firmware that is capable of obtaining and/or analyzing data relating to one or more body parameters of the patient relevant to the patient's disorder, (e.g., epilepsy or depression.) Based upon the data obtained by the detection unit 295, the IMD 200 may deliver the electrical signal to a portion of the vagus nerve to treat epilepsy, depression or other disorders. In one embodiment, the detection unit 295 may be capable of detecting a feedback response from the patient. The feedback response may include a magnetic signal input, a tap input, a wireless data input to the IMD 200, etc. The feedback may be indicative of a pain and/or noxious threshold, wherein the threshold may be the limit of tolerance of discomfort for a particular patient. The term “patient parameters” may refer to, but is not limited to, various body parameters, which may in some embodiments involve sensors coupled to the IMD 200.

The IMD 200 may also comprise a diagnostic unit 290. The diagnostic unit 290 may provide for performing various diagnostics, such as lead impedance tests, tests associated with the stimulation signals, system diagnostic tests, etc., which are described in further detail below. Upon receiving instructions from the external unit 270, the IMD 200 may perform various diagnostics tests as directed by the diagnostics unit 290 and the controller 210. In one embodiment, examples of the lead impedance test may include verifying the impedance of the lead to determine whether there is a problem in the electrical path defined by the path from the IMD 200, through the leads, through the electrodes, and onto the portion of the patient's body targeted for stimulation. Further tests performed by the diagnostic unit 290 may include verifying automatic capture of various patient data, verifying proper reception of data via sensing electrodes, verifying nerve activity, verifying the integrity and characteristics of stimulation signals, verifying whether particular portions of a nerve (e.g., the A-fiber, the B-fibers and/or the C-fiber) are being adequately targeted. This determination may be made by examining the data detected by one or more sensors, (e.g., a sensor electrode in communication with the IMD 200).

The external unit 270 may be an ED that is capable of programming various modules and electrical signal parameters of the IMD 200. In one embodiment, the external unit 270 is a computer system capable of executing a data-acquisition program. The external unit 270 may be controlled by a healthcare provider, such as a physician, at a base station in, for example, a doctor's office. In alternative embodiments, the external unit 270 may be controlled by a patient in a system providing less control over the operation of the IMD 200 than another external unit 270 controlled by a healthcare provider. Whether controlled by the patient or by a healthcare provider, the external unit 270 may be a computer, preferably a handheld computer or PDA, but may alternatively comprise any other device that is capable of electronic communications and programming, e.g., hand-held computer system, a PC computer system, a laptop computer system, a server, a personal digital assistant (PDA), an Apple-based computer system, etc. The external unit 270 may download various parameters and program software into the IMD 200 for programming the operation of the IMD, and may also receive and upload various status conditions and other data from the IMD 200. Communications between the external unit 270 and the communication unit 260 in the IMD 200 may occur via a wireless or other type of communication, represented generally by line 277 in FIGS. 2A and 2B. This may occur using, e.g., wand 155 (FIG. 1) to communicate by RF energy with a generator 110. Alternatively, the wand may be omitted in some systems, e.g., systems in which external unit 270 operates in the MICS bandwidths.

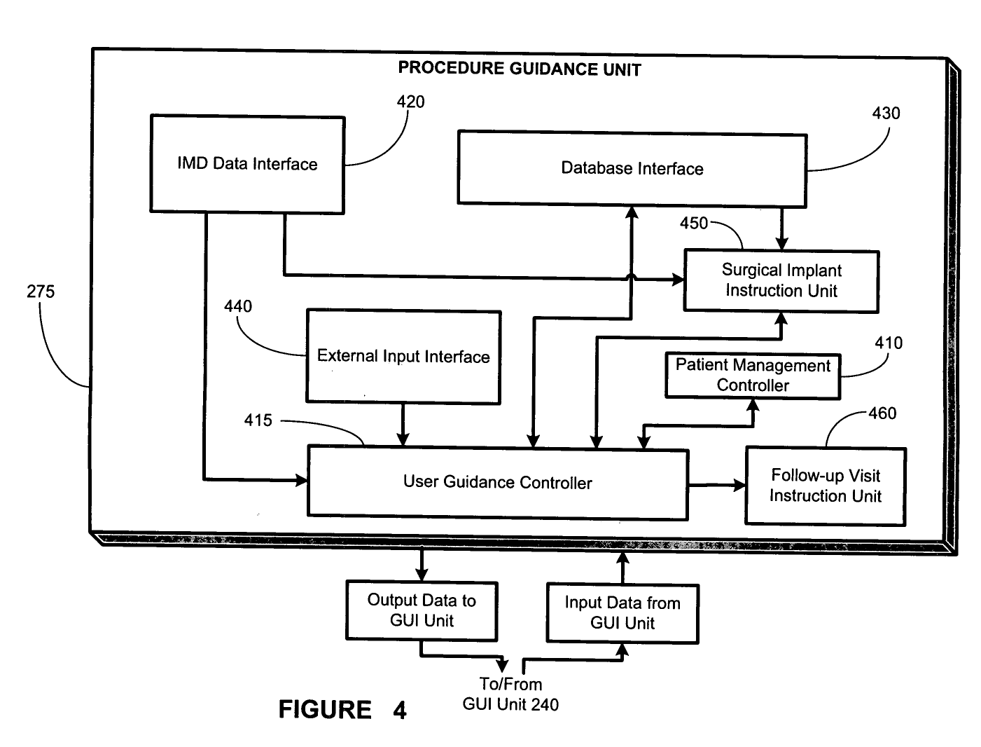

The external unit 270 may comprise a procedure guidance unit 275. The procedure guidance unit 275 is capable of performing various instruction generating processes described herein. The procedure guidance unit 275 is capable of acquiring, storing and/or processing data relating to pre-operation, operating room, post-operation and follow-up office visits for prompting a medical professional to perform various tasks. The procedure guidance unit 275 is capable of providing guidance to a medical professional to perform various tests and/or trouble-shooting steps during pre-operation and operation/implantation of the IMD 200. Further, various information resulting from the analysis performed by the procedure guidance unit 275 may be provided in a text or in a graphical format. Display of the overall data relating to the analysis performed by the procedure guidance unit 275 may be provided to a user to prompt the user to perform various tasks. Based upon performance or non-performance of the task by the user, further analysis and instructions may be provided to the user. This cycle of instructions may be provided until the proper conclusion of the procedure, (i.e., implantation, or follow-up analysis of the operation of the IMD 200 is performed). In other words, the procedure guidance unit 275 is capable of generating data for displaying a series of navigational displays for guiding an operation relating to the IMD 200. A more detailed description of the procedure guidance unit 275 is provided in FIG. 4 and accompanying description below.

In one embodiment, the external unit 270 comprises a graphical user interface (GUI) unit 240. It will be appreciated that the GUI unit 240 may also be a separate unit from the external unit 270. Regardless of whether the GUI unit 240 is part of, or separate from, the external unit 270, the external unit 270 is capable of driving various displays on the GUI unit 240. In one embodiment, the GUI unit 240 is capable not only of receiving data from the external unit 270 for driving one or more displays, but also of receiving inputs from a user, such as a physician or patient, and transmitting the data to the procedure guidance unit 275. The GUI unit 240 may be comprised of a variety of devices, including, but not limited to, a computer terminal, a cathode ray tube (CRT) device, a liquid crystal device (LCD) module, a plasma-display device, etc. The GUI unit 240 may include a touch sensitive screen monitor that is capable of detecting an external input from the user. It may also be a hand-held device, such as a personal digital assistant (PDA), a pen input device, a portable computer device, etc.

In one embodiment, the external unit 270 also comprises a remote unit interface 285. The remote unit interface 285 may contain circuitry for facilitating communications with a remote device 292 via a communication link 297. In one embodiment, the remote device 292 may be a computer system. The communications link 297 may be a wireless or wired communications line, (e.g., a network connection, a wireless connection, a local area network connection (LAN), a wide-area network connection (WAN), a “hotspot” connection, an intranet connection, an Internet connection, a Bluetooth™ link, etc.). A display provided by the GUI unit 240 may be synchronized with a corresponding display on the remote device 292. Using this feature, a remote user may view and/or interact with the GUI displays of the external device. Therefore, using the remote device 292, a remote user may provide additional guidance/feedback to the user of the IMD 200.

In one embodiment, the external unit 270 may comprise a local database unit 255 from which the procedure guidance unit 275 may receive data. Optionally or alternatively, the external unit 270 may also be coupled to a database unit 250, which may be separate from external unit 270 (e.g., a centralized database wirelessly linked to a handheld external unit 270). The database unit 250 and/or the local database unit 255 are capable of storing various patient data. This data may comprise patient parameter data acquired from a patient's body and/or therapy parameter data. The database unit 250 and/or the local database unit 255 may comprise data for a plurality of patients, and may be organized and stored in a variety of manners, such as in date format, severity of disease format, etc. The database unit 250 and/or the local database unit 255 may be relational databases in one embodiment. A physician may perform various patient management functions described below using the GUI unit 240, which may display data from the IMD 200 and/or data from the database unit 250 and/or the local database unit 255. The database unit 250 and/or the local database unit 255 may store various patient data such as seizure types, etc. Inputs into the GUI unit 240 may be sent to the IMD 200 to modify various parameters for the therapeutic electrical signal.

One or more of the blocks illustrated in the block diagram of the IMD 200 in FIG. 2A, may comprise hardware units, software units, firmware units, or any combination thereof. Additionally, one or more blocks illustrated in FIG. 2A may be combined with other blocks, which may represent circuit hardware units, software algorithms, etc. Additionally, any number of the circuitry or software units associated with the various blocks illustrated in FIG. 2A may be combined into a programmable device, such as a field programmable gate array, an ASIC device, etc.

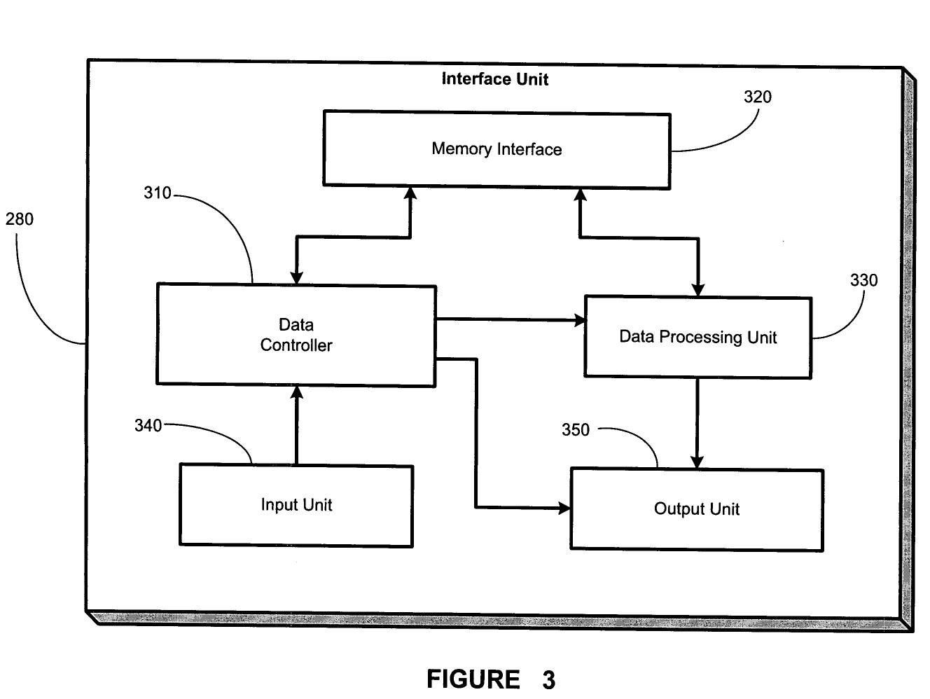

Turning now to FIG. 2B, a block diagram depiction of the IMD 200 is provided, in accordance with an alternative illustrative embodiment of the present invention. The various blocks of FIG. 2B that correspond to similar blocks of FIG. 2A operate in a similar fashion. Additionally, the alternative embodiment of FIG. 2B also comprises an interface unit 280. The interface unit 280 is capable of communicating with the external unit 270. The interface unit 280 may receive instructions and/or data from the external unit 270 via the communication unit 260. The interface unit 280 is capable of various tasks, such as accessing the memory 217 in the IMD 200. Further, the interface unit 280 is capable of providing requested data to the external unit 270 to be displayed by the external unit 270. More detailed description of the interface unit 280 is provided in FIG. 3 and the accompanying description below.

Turning now to FIG. 3, a more detailed block diagram depiction of the interface unit 280 of FIG. 2B, in accordance with one illustrative embodiment of the present invention, is provided. The interface unit 280 may comprise a memory interface 320 that is capable of receiving data from, and/or storing data into the memory 217 of the IMD 200. The memory interface 320 may comprise various hardware and/or firmware objects to facilitate access to the memory 217.

The interface unit 280 also comprises a data controller 310. The data controller 310 is capable of controlling the various functions performed by the interface unit 280, such as receiving and processing information from the external unit 270, as well as providing various parametric data to various portions of the IMD 200. The interface unit 280 may also comprise a data processing unit 330. The data processing unit 330 is capable of processing various patient parameter data and stimulation-related data. For example, upon a command from the external unit 270, the data processing unit 330 may process and correlate patient data with certain therapeutic electrical signal parameters that were used within a pre-determined time period in which the patient data was acquired. For example, after the delivery of a particular therapeutic stimulation cycle, within a predetermined time period, various patient parameters may be collected by the IMD 200. This data may then be correlated and organized in such a fashion that trends relating to the relationship between patient data and various electrical signal parameters may be determined. Statistical and/or other types of data manipulation may also be performed by the data processing unit 330.

Further, the interface unit 280 may also comprise an input unit 340, which is capable of receiving data from the external unit 270 via the communication unit 260. Further, the interface unit 280 may also comprise an output unit 350, which is capable of driving data from the interface unit 280 to the external unit 270. The input unit 340 may comprise various registers, buffers and/or amplifiers to process and streamline data, (e.g., convert data from serial to parallel, or vice versa). The output unit 350 is also capable of registering, buffering and/or amplifying data for transmission from the interface unit 280 to the external unit 270. The interface unit 280 is capable of receiving instructions and providing for various responsive actions in the IMD 200, as well as collecting, processing, and/or storing data. The interface unit 280 provides the ability for using a graphical user interface to provide interactivity between an external user (e.g., a physician), and the IMD 200.

FIG. 4 illustrates a more detailed block diagram depiction of the procedure guidance unit 275 of external unit 270 (FIG. 2), in accordance with one illustrative embodiment of the present invention. The procedure guidance unit 275 may include a user guidance controller 415 that is capable of performing the various procedure guidance functions described herein. The user guidance controller 415 may be a microprocessor, a firmware object (e.g., a field programmable gate array (FPGA) or an ASIC device), and/or a software module. The user guidance controller 415 is capable of extracting data from the database unit 250 and/or local database unit 255 (FIG. 2) via a database interface 430. The database interface 430 may contain various amplifiers, buffers, registers and/or software modules to retrieve and/or send data from the database unit 250 and/or local database unit 255. The procedure guidance unit 275 may also comprise an IMD data interface 420 that is capable of receiving from or sending data to the IMD 200. The IMD data interface 420 may comprise various amplifiers, buffers, registers and/or software modules to send data to the IMD 200 and/or receive data from the IMD 200. The user guidance controller 415 may then provide data for display as part of the GUI unit 240 (e.g., a graphical user interface). The user guidance controller 415 may also receive data from the GUI unit 240 as input from an external user (e.g., a physician input).

The procedure guidance unit 275 may also comprise an external input interface 440. The external input interface 440 is capable of receiving data from a user, (e.g., a physician). Input from a user, via the external input interface 440, may prompt the procedure guidance unit 275 to perform calculations for providing various recommended steps for performing a surgical implantation of the IMD 200, defining therapeutic electrical signals during a follow-up visit, etc. The external input interface 440 may receive technical data relating to specific therapy parameters that are entered by a user/physician. For example, the particular charge required for a therapeutical electrical signal may be input by a physician and received by the external input interface 440.

The procedure guidance unit 275 is capable of interrogating an IMD 200. In one embodiment, interrogating an IMD 200 may refer to communicating with the IMD 200 and receiving various responsive data from the IMD 200. The responsive data from the IMD 200 may include, but is not limited to, various status data, diagnostic and/or test results, parameter value data, etc. The responsive data from the IMD 200 may be referred to as “interrogative signal.” As a result of the interrogation performed by the procedure guidance unit 275, the IMD data interface 420 and the database interface 430 may receive various external data sets that may be used to generate the instructions for the user. The external data received may include data relating to various diagnostic steps performed on the IMD 200, therapy parameter settings used in previous therapeutic electrical signals, type of disease being treated, etc.

The user guidance controller 415 is capable of determining the steps in which a series of procedures relating to implanting or preparing to implant (i.e., pre-operation procedure) an IMD 200 into a patient. The user guidance controller 415 is capable of analyzing input from the external user, (e.g., from a medical professional), to determine various instructions that may be provided to the user to perform a subsequent step. This may include extracting data from a local database unit 255 and/or the database unit 250 to provide guidance to the external user via text, graphics, video, etc. Data may be received via the GUI unit 240, while instructional displays may be sent to the GUI unit 240.

The procedure guidance unit 275 also comprises a surgical implant instruction unit 450, a patient management controller 410, and a follow-up visit instruction unit 460. The user guidance controller 415 is capable of receiving data from a surgical implant instruction unit 450. The surgical implant instruction unit 450 is capable of determining a series of steps that may be performed during a pre-operation, operation and/or a post-operation procedure. The surgical implant instruction unit 450 is capable of determining the consequence of various inputs from the user and/or data from test results, (e.g., impedance test, system test, etc.) and providing an appropriate, responsive instruction(s).

Further, the user guidance controller 415 is capable of providing instructions relating to follow-up visits to an external user. For example, during a follow-up visit with a medical professional, the follow-up visit instruction unit 460 may receive various inputs from an external unit 270 and/or from the IMD 200 and determine an appropriate responsive step that should be taken. For example, the follow-up visit instruction unit 460 may instruct a user, via the external unit 275, to check the stimulation signal parameters and perform an extraction of stimulation data results to determine whether the efficacy of the current parameters is acceptable. Other instructions may be provided by the follow-up visit instruction unit 460 based upon test results, performance test data from the IMD 200, and/or instructions or questions or inquiries from the external user. The user guidance controller 415 may provide the ability to perform a patient management of the IMD 200 and the patient and then prompt the external user to do so. The patient management controller 410 is capable of performing various patient management functions, as described in U.S. patent application Ser. No. 11/495,471, entitled “Patient Management System For Treating Depression Using An Implantable Medical Device,” in U.S. patent application Ser. No. 11/588,702, entitled Patient Management System For Treating Epilepsy Using An Implantable Device,” and in U.S. patent application Ser. No. 11/588,700, entitled “Patient Management System For Providing Parameter Data For An Implantable Medical Device,” wherein all three patent application are hereby incorporated by reference in full.

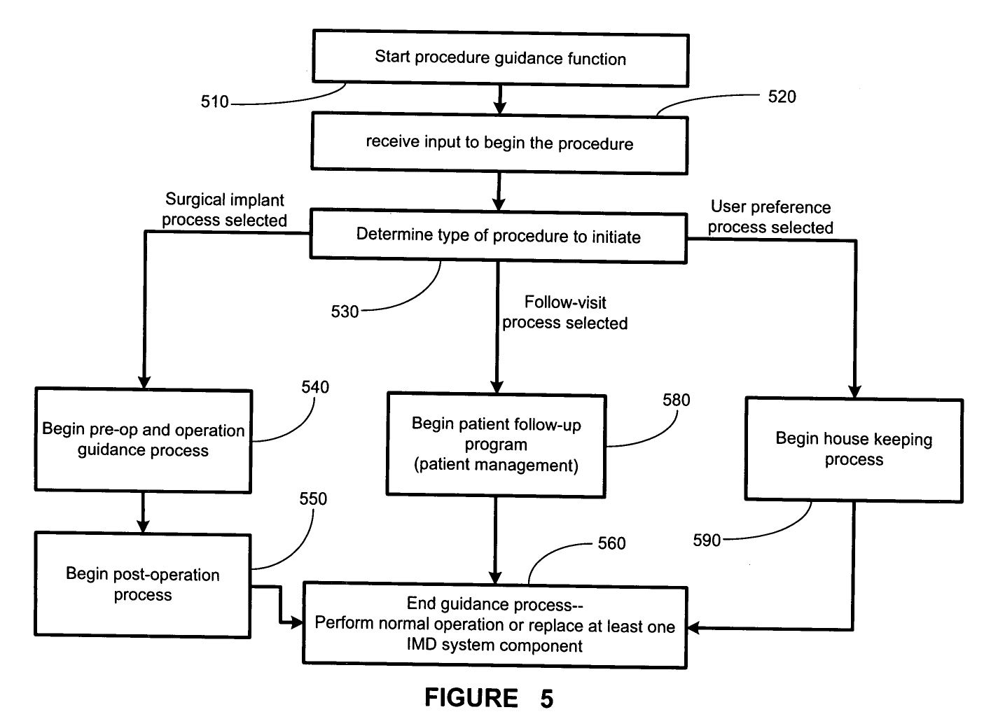

Turning now to FIG. 5, a flowchart depiction of one embodiment of providing guidance for a particular procedure relating to an IMD 200, in accordance with one embodiment of the present invention, is provided. The implantable medical device system described herein may start a procedure guidance function (block 510). This procedure may be initiated by a user, the external device 270, and/or the IMD 200. In one embodiment, the external device 270 may receive an input from the user to begin the procedure guidance process (block 520). Based upon this input, the external unit 270 may make determination as the type of procedure (i.e., surgical implant process, follow-up visit process or user preference process) to initiate (block 530). If a surgical implant process has been selected, a pre-operation and operation guidance process may be initiated (block 540). Various descriptions and exemplary GUI displays associated with the pre-operation and operation guidance process are described in various drawings and accompanying descriptions below. After the pre-operation and the operation guidance processes are completed, the external unit 270 may perform a post-operation guidance process 550. This may include instructing the user to perform various diagnostic tests, and report back the results. Upon conclusion of the post-operation guidance process, the external unit 270 may end the guidance process (block 560). This process may include continuing performing normal operations, or alternatively, if the troubleshooting process has not yet been acceptably resolved, replace one or more portions of the implantable medical device system.

Also, upon receiving input to begin the guidance process of block 520, the external unit 270 may also determine (block 530) that the follow-up visit guidance process has been selected. Upon such a determination, the external unit 270 may perform a patient follow-up guidance process (block 580). The patient follow-up guidance process is described herein, including exemplary GUI displays and accompanying description. Upon completion of the patient follow-up guidance process, the guidance process may then be terminated (block 560).

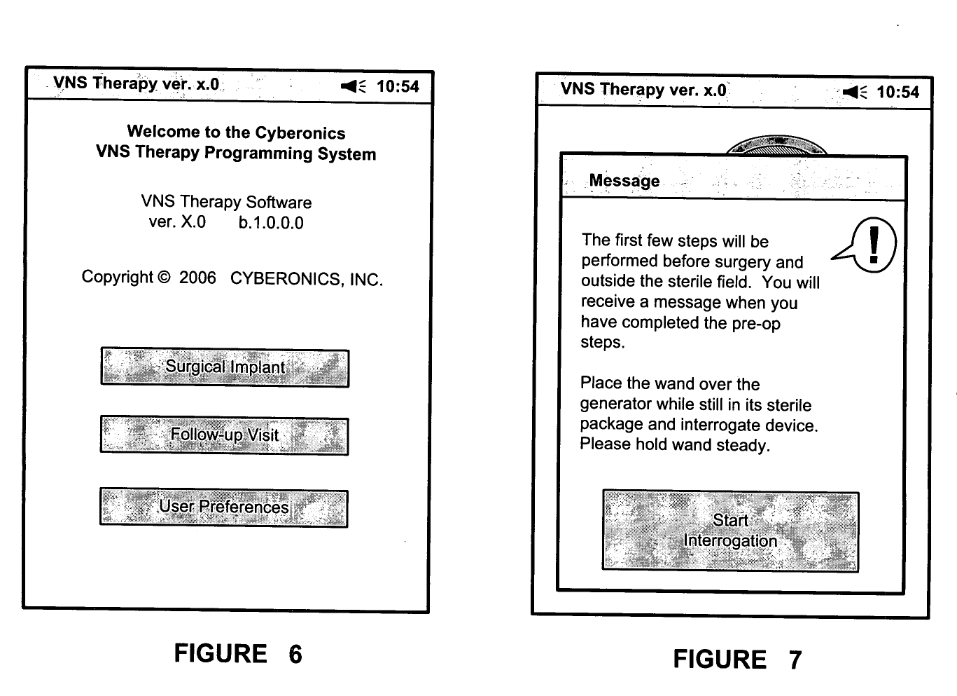

Similarly, referring back to blocks 520 and 530, based upon the input received by the external unit 270, a determination is made that the input relates to a request to initiate a user preference process. The selections represented by the input to the external unit 270 (block 520) and the process determinations (blocks 530) are exemplified in a sample GUI display in FIG. 6 and accompanying description below. Based upon the user preference process being selected, a user preference (i.e., housekeeping) process may be performed (block 590). Upon the conclusion of the user preference process, the guidance process may then be concluded (block 560). The processes described in FIG. 5 are exemplified in various exemplary GUI displays and accompanying description below.

FIGS. 6-44 illustrate various screens (e.g., GUI displays) that may be displayed by the GUI unit 240 of external unit 270 (FIGS. 2A, 2B) for performing various procedure guidance functions for an IMD, in accordance with one illustrative embodiment of the present invention. The screen displays in FIGS. 6-44 are exemplary interactive screens; however, those skilled in the art would appreciate that a variety of other screens may be used with the procedure guidance function embodiments provided herein and remain within the spirit and scope of the present invention. The GUI displays illustrated in FIGS. 6-44 may be integrated into a display device, a computer system, or a variety of types of electronic devices.

In one embodiment, each of the GUI displays described in FIGS. 6-44 may prompt the next action that is to be performed by a user, (e.g., a physician). The interactive navigation/guidance system of the present invention provides for performing various trouble-shooting and/or guided follow-up analysis of the operation of the IMD 200. Based upon the input received by the external unit 270, a subsequent GUI display may then be displayed to prompt the user to perform the next suggested or recommended action. In one embodiment, the external unit 270 (e.g., a hand-held external device) may be sterilized to be used in a sterile field in a pre-operation environment before the IMD 200 is implanted into the patient's body.

When the guidance system provided by the embodiments of the present invention is activated, an initial GUI display screen may be launched to allow the user to select one of several choices, such as a surgical implant procedure selection, a follow-up office visit selection, or a user preference selection. As exemplified in the GUI display screen illustrated in FIG. 6, upon a selection by the user for the surgical implant, a surgical procedure guidance program is launched wherein the surgical implant instruction unit 450 (FIG. 4), in conjunction with the user guidance controller 415, provides various GUI screen prompts. In alternative embodiments, the screen prompts may be accompanied by vocal queues.

FIG. 6 illustrates a GUI screen that provides information relating to various guidance functions concerning an IMD 200 implanted in a particular patient's body, wherein the GUI screen may provide for interactive inputs. The screen illustrated in FIG. 6 provides a program patient data “screen” or GUI display, which illustrates various tasks that may be performed by the procedure guidance system by embodiments of the present invention. The screen/GUI display of FIG. 6 illustrates the type of therapy software being employed, the version, etc. Further, one of several options may be selected to perform a particular task. For example, the GUI display of FIG. 6 provides for selecting one of a “Surgical Implant” process, a “Follow-up Visit” process, and a “User Preferences” process. Other processes may also be added to the display of FIG. 6 and remain within the sprit and scope of the present invention.

FIG. 7 illustrates another exemplary screen that may be driven by the patient management unit 275 and displayed by the GUI unit 240 (FIGS. 2A, 2B) following selection of the “surgical implant” procedure guidance button in FIG. 6. The GUI screen of FIG. 7 may provide a message indicating that a few first steps may be performed prior to surgery outside a sterile field and a new message may then be delivered after the pre-op steps are completed. The screen of FIG. 7 screen also prompts a user to perform the first of those steps, namely, placing a communication tool, such as the wand coupled to a hand-held external unit 270 described above, proximate to the IMD 200 to allow the external unit 270 to interrogate the IMD 200, while the latter is still in its sterile package. Upon properly locating the communication device in proximity to the IMD 200, a user may activate the “Start Interrogation” virtual button depicted in FIG. 7 (or a similar button) to perform an interrogation function. The interrogation process establishes communication between the IMD 200 and the external device 270 (FIGS. 2A, 2B) to receive various data inputs from the IMD 200. The user may begin this process by depressing the “Start Interrogation” button illustrated in FIG. 7. In an alternative embodiment, an additional “button” may be added to the screen of FIG. 7 to prompt a demonstration of a screen video or animation exemplifying the placing of the wand/external unit 270 over the package in which the sterile IMD 200 resides.

Upon selection of the “Start Interrogation” GUI button in FIG. 7, a subsequent GUI display screen may be launched, as exemplified in FIG. 8. In FIG. 8, various parameter settings are foreshadowed by a message prompting the user to confirm the model and the serial number of the IMD 200 being implanted. The message of FIG. 8 also prompts the user to program the patient's initials, the indication (i.e., the medical condition for which the patient is to be treated by the IMD), the implantation date, the lead model and serial numbers, etc., on the next screen. The “program patient data” button is provided to initiate the programming of the patient data into the system.

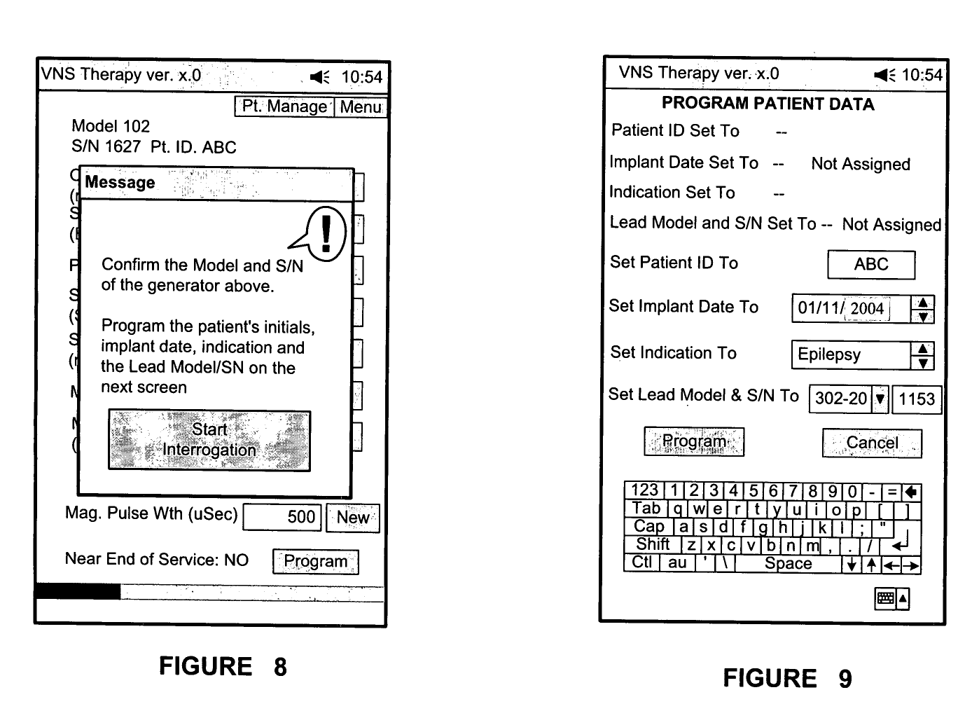

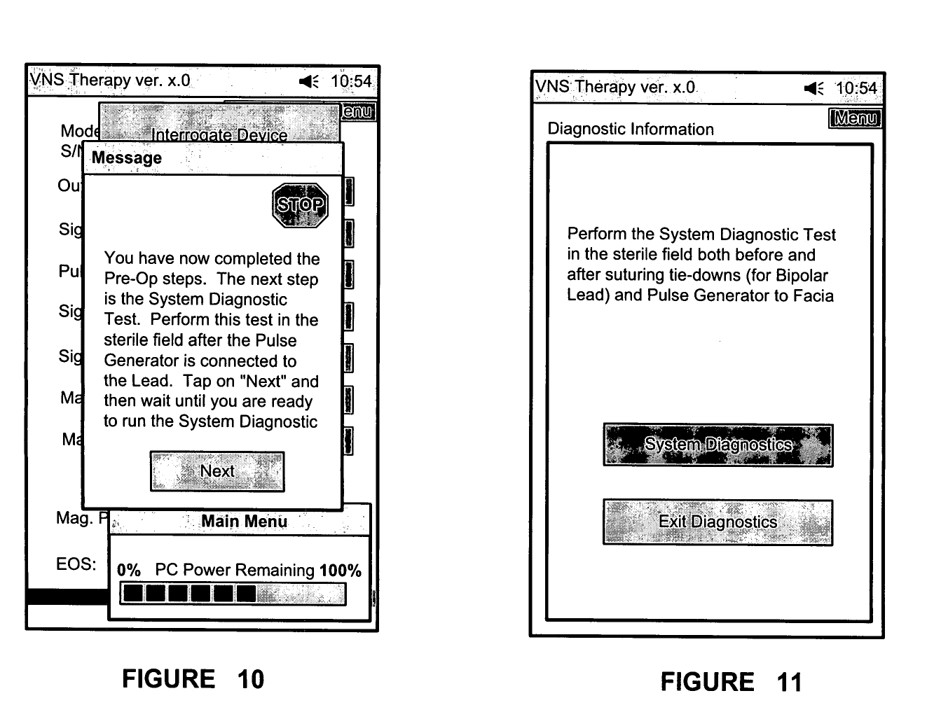

Continuing with the pre-operation process, FIG. 9 illustrates the result of selecting the “program patient data” button of FIG. 8. This GUI screen allows for entry and programming of the information referred to in FIG. 8, such as the model number and serial number of the IMD 200, the lead model and serial number, etc. An interactive keyboard, as illustrated in FIG. 9, may be used to enter the various information, such as patient ID number, IMD implant date, treatment indication, lead serial and model numbers, etc. Upon completion, the user may depress or activate the “Program” GUI button of FIG. 9, which may prompt the subsequent screen illustrated in FIG. 10.

Turning now to FIG. 10, a message may indicate to the user that the pre-operations steps are complete and that the next step is to perform the system diagnostics test. The message may prompt the user to perform those steps in a sterile field after the IMD 200 is connected to the lead. The “Next” GUI button may be selected to run the system diagnostics test. Upon the selection of the “Next” button of FIG. 10, the GUI screen exemplified in FIG. 11 is then provided.

The GUI screen of FIG. 11 displays a “Diagnostic Information” GUI screen, prompting the user to perform the system diagnostics test in a sterile field, both before and after performing the suturing tie-downs. A “System Diagnostics” GUI button and an “Exit Diagnostics” GUI button are provided. The user may perform the system diagnostics test by depressing the “System Diagnostics” GUI button of FIG. 11. The system diagnostics test may include a lead impedance test. For example, a one milliamp of the current may be provided and, using Ohm's law, the impedance “seen” by the lead may be calculated. In some embodiments, the lead impedance test may be calculated by the IMD 200, in other embodiments, raw data is sent to the ED and calculation is made separate from the IMD 200.

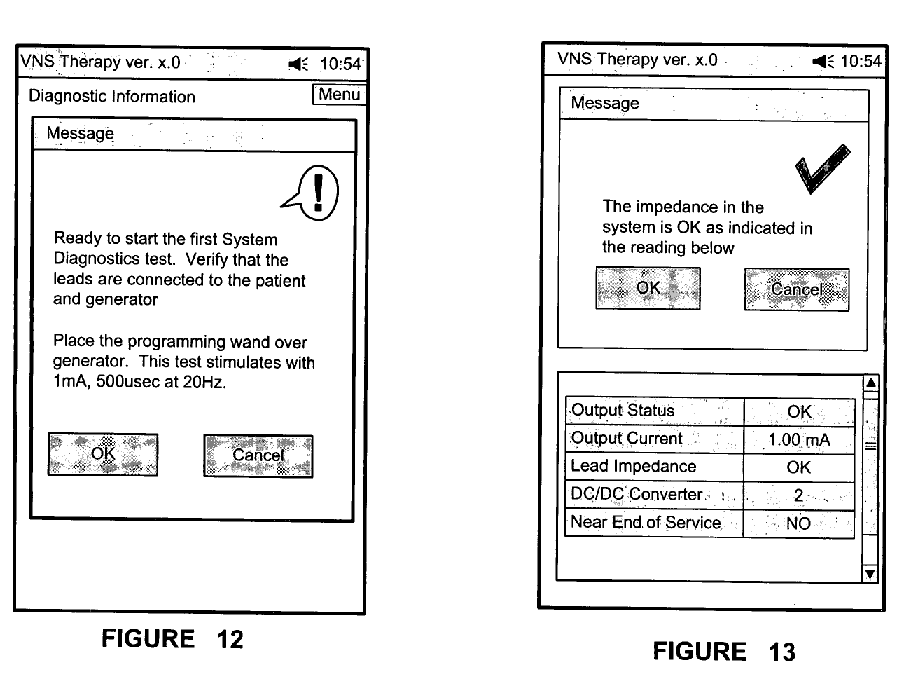

In one embodiment, the external unit 270 may store the initial lead impedance value for later comparison. Further, periodic impedance tests may be performed and recorded and an analysis of the percent change from a particular threshold impedance value may be calculated to determine the change in lead impedance over time. Additionally, the system diagnostics test may include various other types of tests to check the integrity of the implantable medical system. These tests may include, but are not limited to, normal mode test, magnet mode test, pre-implantation test, such as diagnostics relating to the IMD 200 itself, testing sense electrodes, etc. Upon selection of the system diagnostics button of FIG. 11, a subsequent GUI screen may appear as illustrated in FIG. 12.

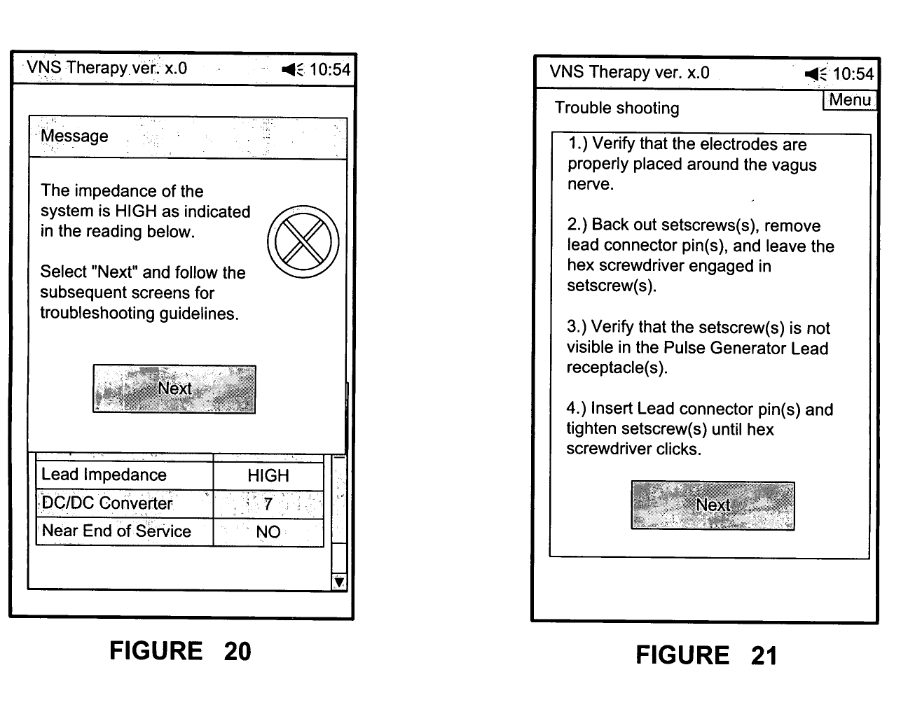

A message in the GUI display screen of FIG. 12 may indicate that the system diagnostics test is ready to be started. A message may prompt the user to perform one or more steps to initiate the diagnostics test, e.g., verify that the leads are connected to the target area of the patient's body, as well as to the IMD 200. In one example, the message may also prompt the user to place the programming wand, (i.e., the external unit 270 or a portion thereof) over the IMD 200 to perform the test. One example of this test may provide for a pulsed signal of 1 milliamp, 500 microsecond, and 20 hertz. Upon the selection or activation of the “OK” GUI button of FIG. 12 (and performing the other steps specified in the on-screen messages), a subsequent exemplary GUI screen, as illustrated in FIG. 13, may be provided by the external unit 270. It should be appreciated that, where multiple electrode pairs are provided (e.g., where separate sensing and stimulation electrodes are used), multiple lead impedance tests may be performed as part of a system diagnostics test. The external unit 270 may provide additional screens (now shown) to prompt the user to ensure that the impedance of all electrodes present are tested. If the diagnostic test of FIG. 12 fails, then subsequent troubles-shooting processes may be performed, as described in FIG. 21-26 and accompanying description below.

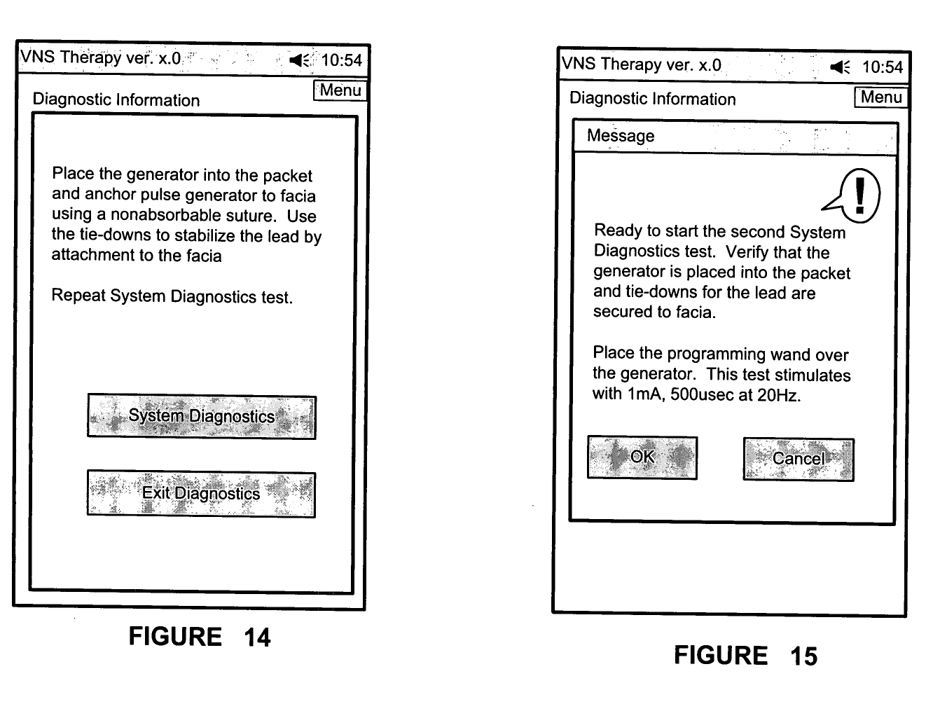

Continuing referring to FIG. 12, after evaluating the result of the system diagnostics test in one embodiment, the external unit 270 may determine that the impedance in the implantable device system is acceptable (which, in some embodiments, may involve multiple tests) and in response provide a message to that effect as indicated in FIG. 13. In alternative embodiments involving multiple electrode pairs, the message may provide an indication of a successful test for each electrode pair. Upon selection of the “OK” GUI button in FIG. 13, a “Diagnostics Information” GUI screen, as exemplified by FIG. 14, may be provided. The user, as indicated in FIG. 14, may then be prompted to place the IMD 200 in the pocket (i.e., the subcutaneous area formed by the surgeon to receive and hold the implanted IMD 200) and anchor it to the facia using non-absorbable suture. The message may also prompt the user to use the tie-downs to stabilize the lead by attachment to the facia. The user may be again prompted to perform the system diagnostics test by depressing the “System Diagnostics” GUI button, as indicated in FIG. 14.

Upon the selection of the “System Diagnostics” GUI button of FIG. 14, a subsequent GUI display screen is provided, as illustrated in FIG. 15. A message, as illustrated in FIG. 15, indicates that the second system diagnostics test is ready to be initiated and provides a test simulation that prepares the IMD to receive a test signal. The message also prompts the user to verify that the IMD 200 is placed into the pocket and the tie-downs for the leads are secured to the facia.

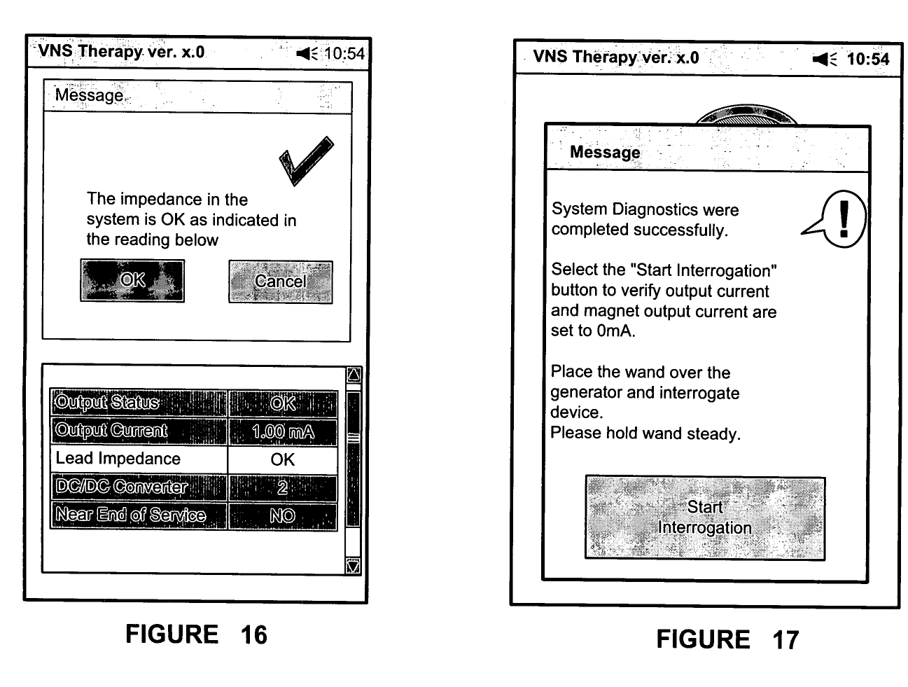

Upon accepting the instruction to perform the second system diagnostics test by depressing the “OK” button of FIG. 15, a message, as illustrated in FIG. 16, may be provided. The message may indicate that the impedance in the system is OK, as indicated in the reading below. The reading below may provide for various status indications, such as output status being acceptable, the output current being 1 milliamp, the lead impedance being acceptable, alternatively including the actual lead impedance measured during the test (not shown in FIG. 16) etc. These indications may be acceptable to the user and the user is invited to depress the “OK” button to move to the next step in the implantation procedure.

Upon completion of the second system diagnostic test, an exemplary GUI screen, as illustrated in FIG. 17, may be provided for confirming the interrogation process and the system diagnostics test. The message may indicate to the user that the system diagnostics were completed. Further, the user may be prompted to select the “Start Interrogation” button to verify that the output current and the magnet output current are actually set to zero milliamps. This may be performed by placing the wand/external unit 270 over the generator and interrogating the IMD 200 by depressing the “start interrogation” button, which causes the IMD 200 to report back to the external unit 270 its programmed settings.

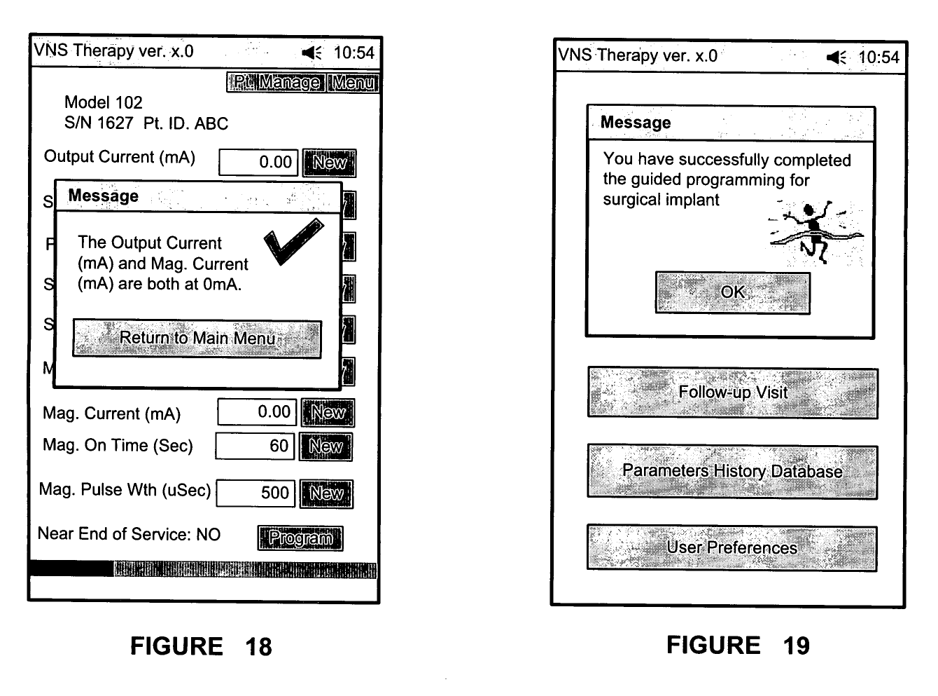

The activation of the “Start Interrogation” GUI button provides for yet another interrogation of the IMD 200. This interrogation may be intended to prompt the user to check that the output current are set to zero milliamp before the patient is released after implantation of the IMD 200. Upon starting the interrogation, an exemplary GUI display screen illustrated in FIG. 18 may be provided. This GUI display screen may include a message that the output milliamp and the magnet current are both at zero milliamps. This interrogation may be concluded by the activation of the “Return To Main Menu” GUI button of FIG. 18.

Upon the selection of the “Return To Main Menu” GUI button of FIG. 18, a subsequent GUI display screen may be provided, as illustrated in FIG. 19, indicating to the user that the implantation process has been completed. A message may indicate that the user has successfully completed the target programming for surgical implant. The user may select the “OK” GUI button, or various other GUI buttons to investigate other procedural instructions by depressing one of several buttons, such as the “Follow-up Visit,” the “Parameter History Database” (to access historical data) and the “User Preferences” (to adjust settings for the external unit 270 itself) GUI display buttons. Upon the displaying of the GUI display screen of FIG. 19, the user receives an indication that the implantation process is complete.

Referring back to FIGS. 14, 15 and 20, the consequence of not properly passing the system diagnostics test is shown. FIG. 20 illustrates the message screen that indicates that the system diagnostics test has not passed after the activation of the “OK” GUI button in FIG. 15. The method displayed in the GUI screen of FIG. 20 indicates that the impedance of the system is high and provides a chart indicating the impedance score/value and providing an indication of battery life. A “Next” GUI button is offered to provide the user with an option to activate a trouble-shooting guidance process.

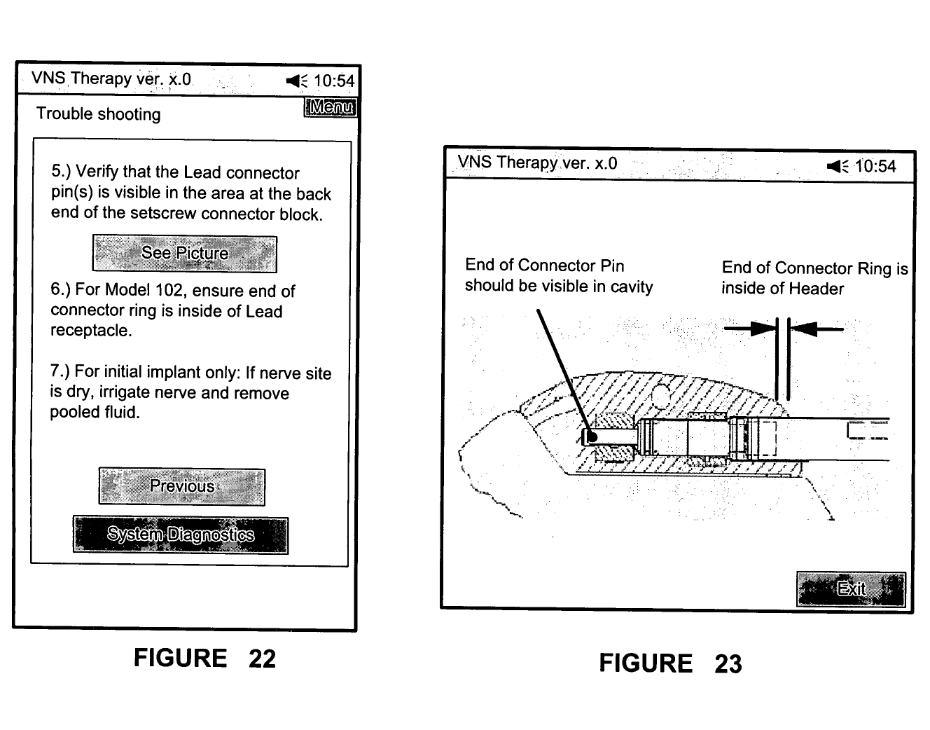

Upon activation of the “Next” GUI button of FIG. 20, a troubleshooting screen is illustrated in FIGS. 21 and 22. The troubleshooting screen may provide various instructions that are to be followed by the user, (i.e., the surgeon). Various steps are indicated in FIGS. 21 and 22 to guide the user through various steps to resolve the high impedance problem. For example, the user may be prompted to verify that the electrodes are properly placed in the targeted areas of the patient's body, (e.g., the vagus nerve or a target sensing area). Further, the user may be prompted to back out the setscrew(s), remove the lead connector pins, and leave the hex screwdriver engaged in the setscrew(s). Referring simultaneously to FIGS. 21, 22 and 23 a visual depiction of the details regarding the setscrew(s) and other portions of the electrodes may be provided. The graphics of FIG. 23 depicts the connector pin of the lead being properly inserted to the header of the IMD 200. The user may be prompted, as indicated in FIG. 21 (step 3), to verify that the setscrew(s) are not visible from the pulse generator lead receptacle.

As shown in FIG. 21 (step 4) the user may be prompted to insert the lead connector pins and tighten the setscrew(s) until the hex screwdriver clicks. Upon performing this step, the user may be prompted to depress the “Next” GUI button to receive further instructions. As indicated in FIG. 22, the user may then be prompted to verify that the lead connector pins are visible in the area at the back end of the setscrew connector block. The user may be given the option to click on the “See Picture” GUI button to prompt the display of the depiction of the connector pin assembly of FIG. 23. The display of FIG. 23 may illustrate one or more variety of illustrations, such as one or more still pictures/images, animations, videos, computer simulations, and/or other type of moving-picture demonstration. The illustration of FIG. 23 may be custom tailored to the specific type of IMD 200 being implanted. Based upon the type of IMD 200 being implanted, the surgical implant instruction unit 450 (FIG. 4) may access, via the database interface 430, the appropriate graphic display to be provided. Therefore, various animations/graphics outputs may be stored in the databases 250, 255 for demonstration during a surgical procedure.

Upon verifying the lead connector pins, more specific instructions, such as prompting the user to ensure that the end of the connector ring is inside the lead receptacle for a particular model of the IMD 200, (e.g., Cyberonics VNS Model 102) is provided. Further, for initial implantation of an IMD 200, if the nerve site is dry, the user is prompted to irrigate the nerve and remove pooled fluid. As illustrated in FIG. 22, upon the completion of trouble-shooting the IMD 200 system, the user is then prompted again to select the “System Diagnostics” GUI button to initiate the diagnostics of the IMD 200. In the event that the system diagnostics fails a second time, as indicated in FIG. 24, the user is prompted with a message indicating that the impedance of the system is high, as indicated in the display of FIG. 24. FIG. 24 shows a status display that indicates that the output status is at LIMIT and the lead impedance is HIGH.

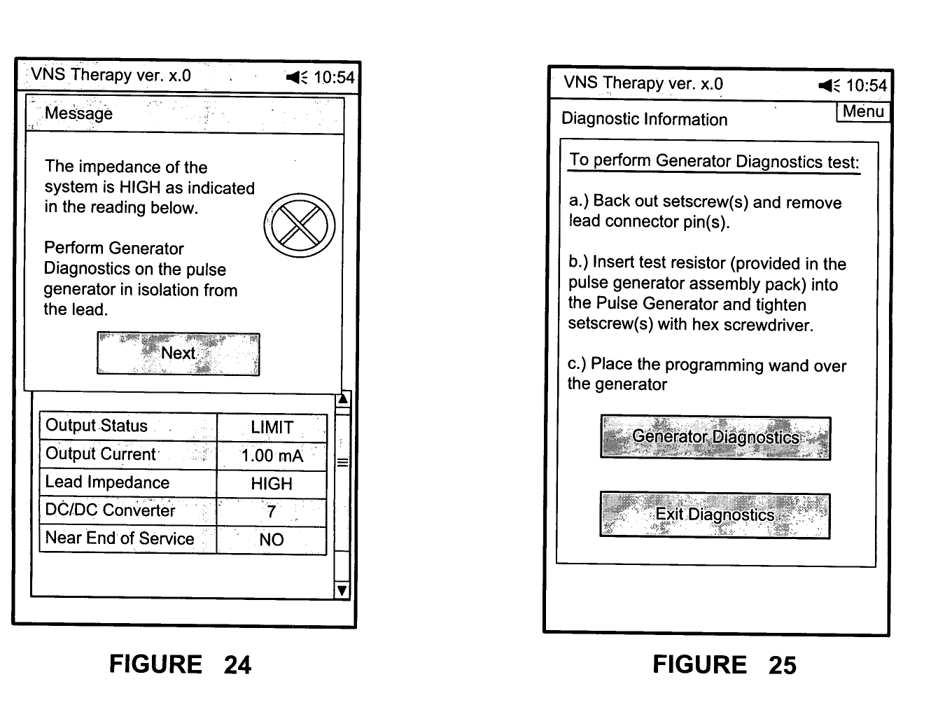

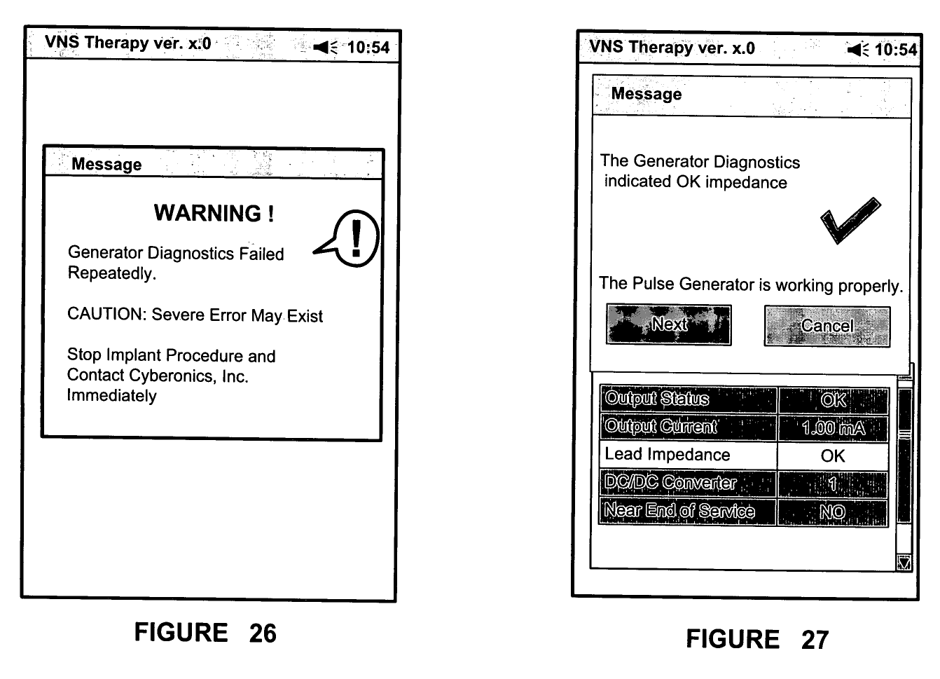

The user is then asked to perform a generator diagnostics on the pulse generator, (e.g., the IMD 200) in isolation of the lead. The user may select this process by activating the “Next” GUI button to perform the generator diagnostics test. Upon selection of the “Next” button illustrated in FIG. 24, an exemplary GUI screen indicated in FIG. 25 is displayed. FIG. 25 illustrates the generator diagnostics test process. The user is prompted to back out the setscrew(s), remove the lead connector pins, insert the test resistor into the IMD 200 and place the programming wand over the IMD 200. The user may then activate the “Generator Diagnostics” GUI button to perform the generator diagnostics. In the event that the impedance continues to be high while using the test resistor, the user is prompted that the test has failed again and is prompted to call Cyberonics, Inc., for further instructions, as indicated in FIG. 26. This series of tests is used to determine whether the problem lies with the leads, the electrodes, or with the IMD 200 itself. Therefore, a failure of the generator diagnostics using a test resistor may indicate that the IMD 200 may be malfunctioning, hence, the prompt to contact Cyberonics, Inc., in FIG. 26.

Upon performance of a successful generator diagnostics, the GUI screen of FIG. 27 may be provided, indicating to the user that the generator diagnostics test shows an acceptable impedance and that the pulse generator (i.e., the IMD 200) is operating properly. The GUI screen of FIG. 27 also indicates that the output status is “OK”, the output current is one milliamp and the lead impedance is “OK”. The status indicated in FIG. 27 may be stored (e.g., into the IMD 200 and/or the databases 250, 255) to keep track of successful diagnostics tests. This data may be useful in conditions where two successful diagnostics are required.

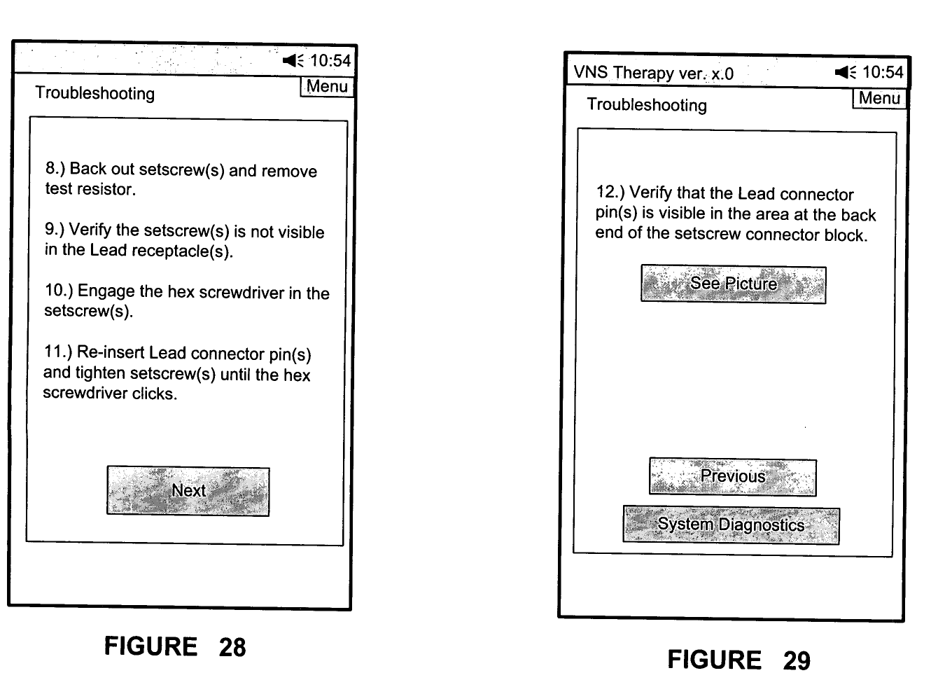

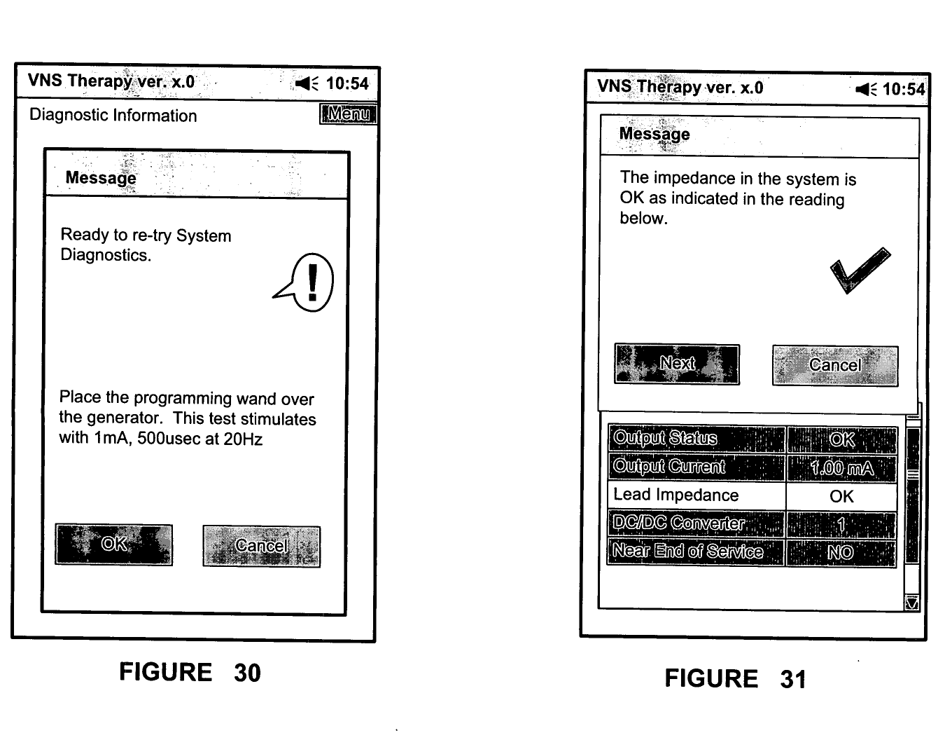

Subsequently, the user is prompted, as indicated in FIG. 28, to back up a setscrew, remove the test resistor and verify that the setscrew is not visible, engage the hex screwdriver in the setscrew to reinsert the lead connector pins, and tighten the setscrew with the hex screwdriver. Upon activation of the “Next” GUI button of FIG. 28, the GUI screen of FIG. 29 may be displayed. The user is prompted to verify that the lead connector pin is visible in an area at the back of the setscrew connector block. FIG. 29 prompts the user to verify the lead connector pins by activating the “System Diagnostics” GUI button of FIG. 29, which provides for the GUI display screen illustrated in FIG. 30, indicating that the system diagnostics is ready to be retried. Upon activation of the “OK” GUI button of FIG. 30, a system diagnostics test is performed and a subsequent message, as indicated in FIG. 31, may be provided indicating that the lead impedance is “OK.” Once the procedure guidance system described herein indicates that the system diagnostics is successful, the nominal procedure path is followed. Therefore, as described herein, in one embodiment, the guidance system may require two successful system diagnostics. If either of the system diagnostics results in a high impedance reading, the user is prompted to resolve the issue by following the trouble-shooting processes described herein.

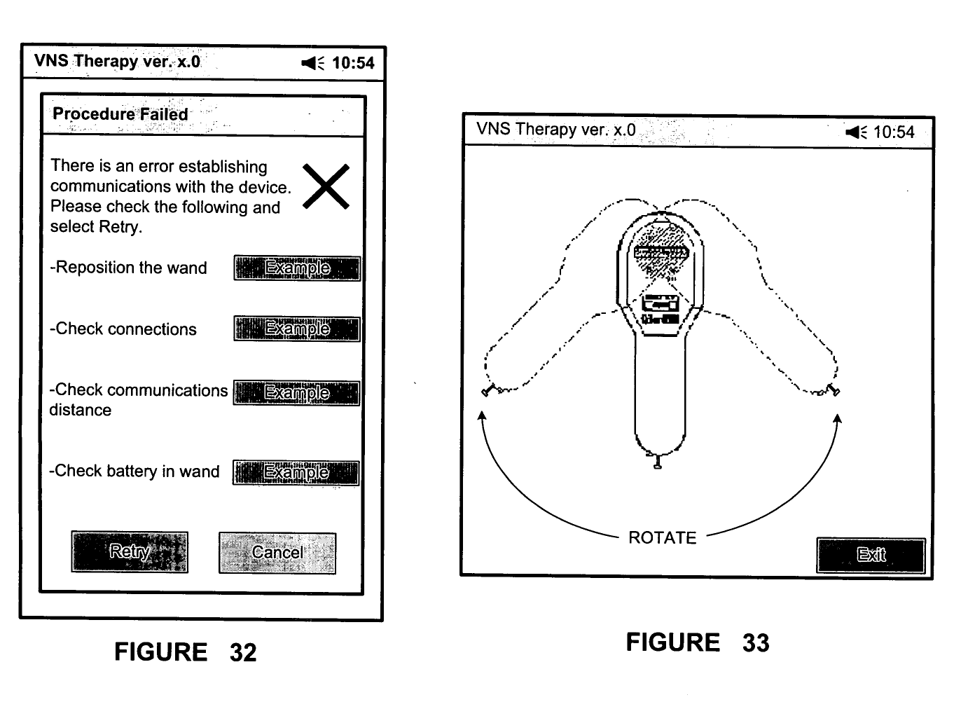

Turning now to FIG. 32, if there are any communications problems between the IMD 200 and the external device, various trouble-shooting guidance screens are provided by the procedure guidance unit 275 of embodiments of the present invention. For example, FIG. 32 illustrates that a communication procedure has failed indicating that there is an error establishing communications between the IMD 200 and the external unit 270. The user is then prompted to perform various tasks, such as repositioning the wand, checking the connections, checking communication distance, checking the battery in the wand, etc., as illustrated in the GUI display of FIG. 32. Further, several “Example” GUI buttons are illustrated. A user may have the option to activate an “Example” GUI button to initiate a graphical display of an example of one of the tasks indicated in FIG. 32. These graphical-display examples are provided in FIGS. 33-36.

FIG. 33 illustrates an animation to assist a user in performing the instruction relating to the repositioning of the wand provided in FIG. 32. The illustration of FIG. 33 may be a still-shot display or an animation that is model-dependent. In other words, if a specific type of wand is being utilized, the graphics animation may be specific to the type of wand being used, which may be determined, preferably automatically by the surgical implant instruction unit 450 (FIG. 4).

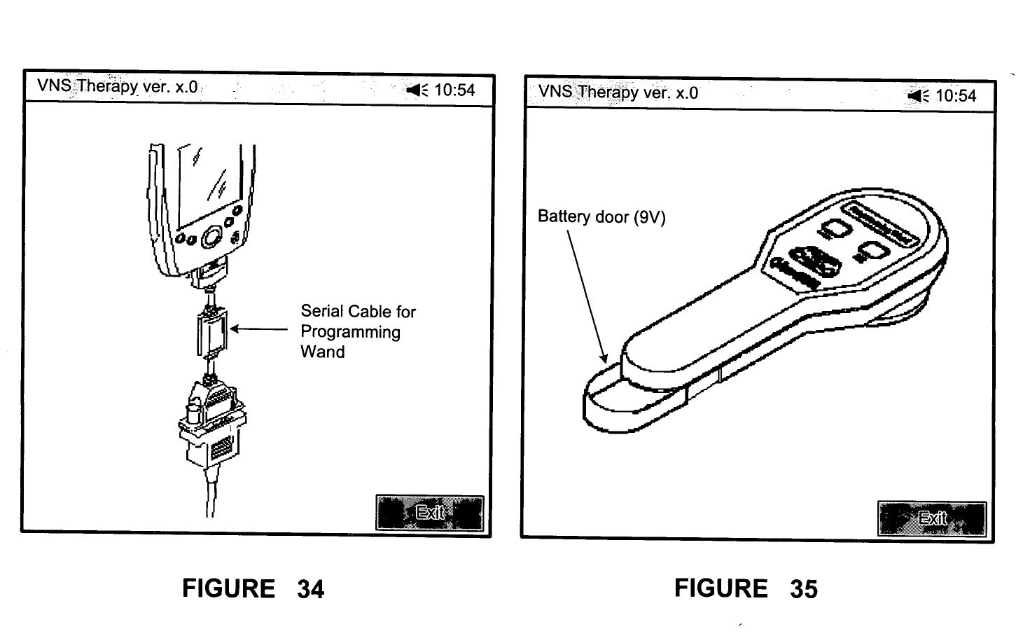

FIG. 34 illustrates a graphical example of the instruction relating to checking connections, as indicated in FIG. 32. The hand-held serial cable for the programming wand and the connections are illustrated to assist the user in performing the checking of the connections. The illustration of FIG. 34 may be custom tailored to the specific wand/hand-held device being used for the implant procedure, and may comprise a still-shot display and/or animation.

FIG. 35 illustrates a graphical example of the instruction relating to checking the battery of the wand, as indicated in FIG. 32. The proper location for accessing the battery is illustrated in FIG. 35. The illustration of FIG. 35 may be custom tailored to the specific wand/hand-held device and/or battery being used during the implant procedure.

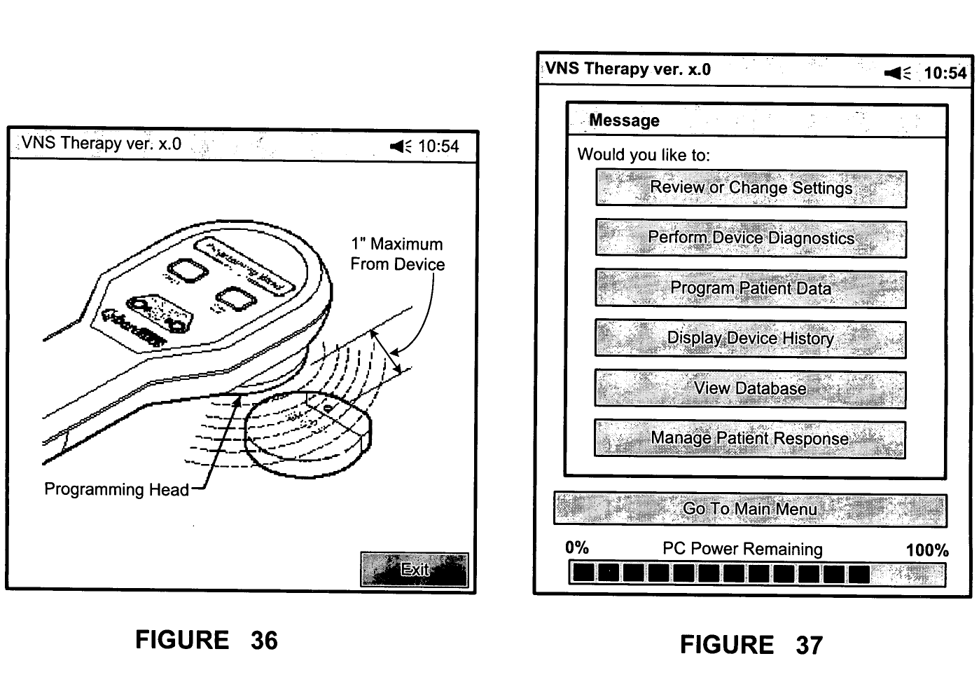

FIG. 36 illustrates a graphical example of the instruction relating to ensuring the distance between the hand-held and wand. In one embodiment, the display of FIG. 36 indicates that the distance between the hand-held and wand should be no greater than a predetermined distance, such as one inch. The illustration of FIG. 35 may be custom tailored to the specific wand/hand-held device and/or battery being used during the implant procedure and/or the physical characteristics of the patient.

The display screens described in FIGS. 33, 34, 35 and 36 may be tailored or customized for particular wands or other components being used for the implant procedure. This customization may be accomplished by utilizing the procedure guidance unit 275 for looking up data in the databases 250, 255 (FIG. 4). The user may perform the various trouble-shooting steps provided by the navigated screens described above to perform trouble-shooting and proper installation of the IMD 200 into the patient's body. The screens described herein may be automated by the procedure guidance unit 275 to automatically select various instructions to be performed by the user based upon the input received by the external unit 270.

Referring simultaneously to FIG. 6 and FIG. 37, upon the selection by the user of the “Follow-up Visit” GUI button of FIG. 6, the external unit 270 may enter into a follow-up visit guidance mode. Upon entering the follow-up visit guidance mode, a GUI display of the type indicated in FIG. 37, may be provided. A message prompts the user to make one of various selections that include: review or change settings, perform device diagnostics, program patient data, display device history, view database, manage patient response, or go to the main menu. Generally, a follow-up visit may consist of various steps, such as interrogation of the IMD 200, checking and adjusting parameters based on titration, efficacy, and/or side effects. The follow-up visits may also require performing diagnostics to ensure that the IMD 200 is functioning as intended, has an adequate battery life remaining, and to ensure that the predetermined therapy is being delivered.

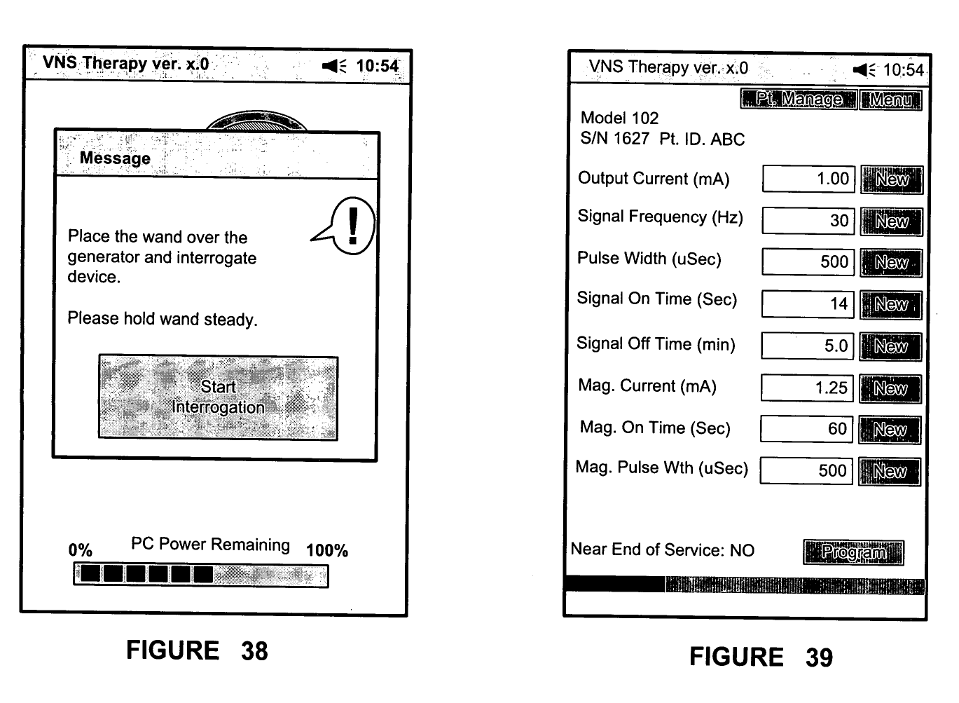

Further, additional interrogation may be made to ensure that the parameters defining the stimulation signal are programmed into the IMD 200 based upon various factors, such as titration, efficacy and/or side effects. Based upon the follow-up visit screen, if the user activates the “Review of Change Settings” GUI button illustrated in FIG. 37, the user, as described in FIG. 38, may be prompted to start interrogation. The user may be prompted to ask to place the wand over the IMD 200 to perform the interrogation, as indicated in FIG. 38. FIG. 39 illustrates an exemplary screen that may result from the selection of the “Review or Change Setting” GUI button of FIG. 37. The user is then asked to begin the interrogation of the IMD 200 by activating the “Start Interrogation” GUI button of FIG. 38.

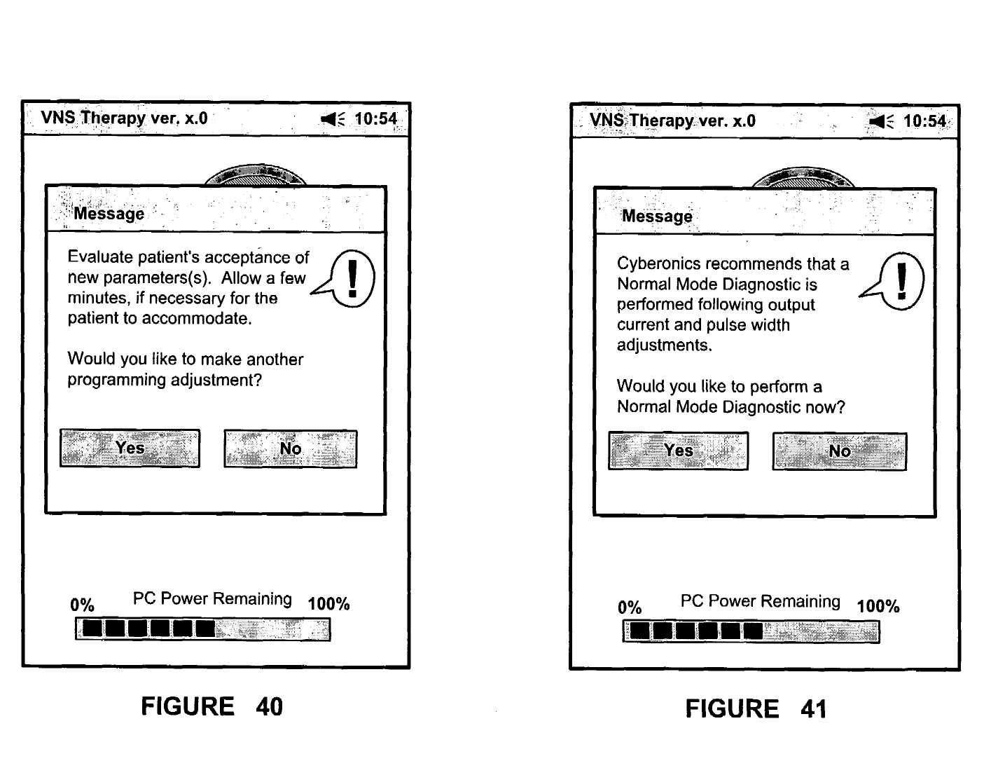

Upon completion of the interrogation, the parameter entry screen illustrated in FIG. 39 is displayed. The GUI display of FIG. 39 may be used to enter/change various parameters relating to delivering therapy. In one embodiment, a number of additional patient management screens may be provided to perform patient management and/or adjustment of stimulation therapy. These patient management display screens may be similar to the GUI display screen illustrated in U.S. patent application Ser. No. 11/588,700, entitled “Patient Management System For Providing Parameter Data For An Implantable Medical Device.” Based on the activation of the “Program” GUI button of FIG. 39, a GUI display screen illustrated in FIG. 40 may prompt the user to evaluate the patient's tolerance of the new parameters and either accept or adjust the parameters. The GUI display screen of FIG. 40 prompts the user to allow a few minutes for the patient to respond to the new adjustments. If the user activates the “NO” GUI button, the message screen of FIG. 41 is provided. FIG. 41 indicates that there is a recommendation to perform a normal mode diagnostics test if any adjustments have been made to the output current and pulse width of the stimulation signal. This is provided because of the importance of determining the effects of any parameter adjustments on the generator's ability to deliver program stimulation given the capabilities of the battery, the lead and/or the nerve impedance. If the procedure guidance unit 275 (FIG. 4) determines that the combination of the stimulation parameters selected may not be appropriate for the IMD 200 to perform, a message (not shown) may be sent to the user that the current parameter setting may be undesirable.

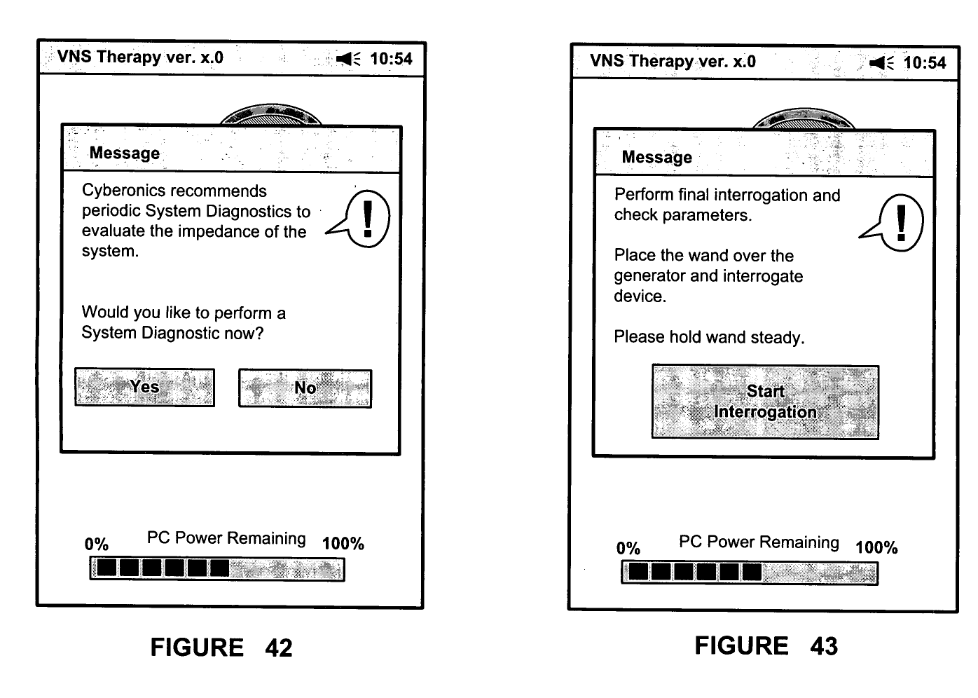

Upon successful completion of a normal mode diagnostics test, a message prompting the user to perform a system diagnostics upon test is provided to the user, as illustrated in FIG. 42. The user has the option to accept or decline to perform the system diagnostics test by activating the “YES” GUI button or the “NO” GUI button, respectively. The system diagnostics test may be used to test the impedance of the system. The user then is prompted to start another interrogation of the IMD 200, as indicated by the GUI screen of FIG. 43. This interrogation may be performed to allow the user to verify whether the parameters programmed by the user were successfully received and implemented by the IMD 200.

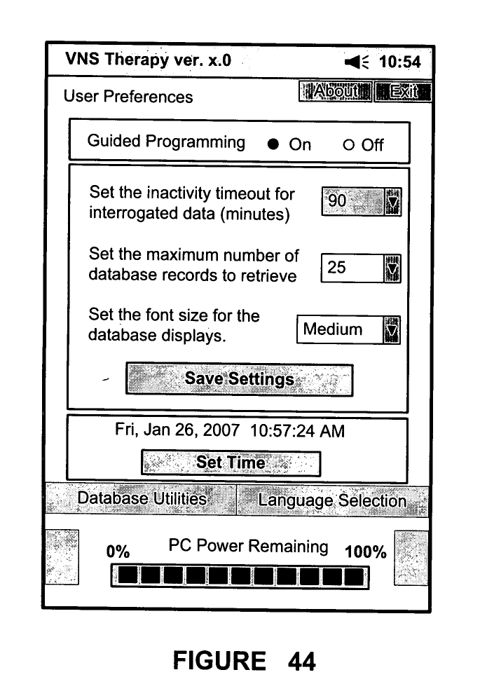

FIG. 44 illustrates a subsequent GUI display screen that may be displayed following selection of the “User Preferences” button of FIG. 6. The screen of FIG. 44 allows various settings for the external unit 270 to be selected. For example, as shown, the guided procedure function provided by embodiments of the present invention may be turned on or off. Various options, such as setting the inactivity time-out for interrogating data may be programmed. Further, the maximum number of database records to retrieve may be programmed and other controls such as the display size for database displays may also be programmed. Therefore, the user is capable of turning on or off the guided programming described by embodiments of the present invention.

Utilizing embodiments of the present invention, guided and/or automated feedback to the user may be provided based upon input from the user and/or the IMD 200. The external device may provide various instructions to the user in performing surgical implants, follow-up visits, and/or parameter adjustments.

All of the methods and apparatuses disclosed and claimed herein may be made and executed without undue experimentation in light of the present disclosure. While the methods and apparatus of this invention have been described in terms of particular embodiments, it will be apparent to those skilled in the art that variations may be applied to the methods and apparatus and in the steps, or in the sequence of steps, of the method described herein without departing from the concept, spirit, and scope of the invention, as defined by the appended claims. It should be especially apparent that the principles of the invention may be applied to selected cranial nerves other than, or in addition to, the vagus nerve to achieve particular results in treating patients having epilepsy, depression, or other medical conditions.

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed:

1. A method for guiding a medical procedure relating to an implantable medical device operatively coupled to a cranial nerve, comprising:
establishing communications between said implantable medical device and an external device;
performing an implant procedure for implanting said implantable medical device;
performing a first diagnostic process of said implantable medical device;
receiving with said external device a first signal from said implantable medical device based on said first diagnostic process; and
displaying a first instruction using said external device based upon said first signal received by said external device, said first instruction comprising information relating to guiding said implant procedure.

2. The method of claim 1, wherein performing said first diagnostic process comprises performing a first impedance test.

3. The method of claim 2, wherein displaying said first instruction comprises displaying an instruction to insert the implantable medical device into a portion of a patient's body and establish lead connections.

4. The method of claim 1, further comprising:
performing impedance test;
receiving with said external device a second signal from said implantable medical device indicative of the result of said impedance test;
determining whether said impedance test was passed;
performing an electrical diagnostic test to test an output current in response to determining that said impedance test was passed;
determining whether said electrical diagnostic test has passed; and
providing a display indicating that said electrical diagnostic test has passed.

5. The method of claim 1, further comprising:
performing impedance test;
receiving with said external device a second signal from said implantable medical device indicative of the result of said impedance test;
determining whether said impedance test was passed;
performing an electrical diagnostic test to test an output current in response to determining that said impedance test was passed;
determining whether said electrical diagnostic test has passed; and
providing a display relating to performing at least one of a lead-check and a lead-adjustment, in response to a determination that at least one of said impedance test and said electrical diagnostic test was not passed.

6. A method guiding a medical procedure relating to an implantable medical device operatively coupled to a cranial nerve, comprising:
establishing communications between said implantable medical device and an external device;
performing a diagnostic process on said implantable medical device;
receiving with said external device a first signal from said implantable medical device as a result of said diagnostic process; and
displaying at least one selectable option using said external device based upon said signal received by said external device, said selectable option relating to an operational setting of said implantable medical device.

7. The method of claim 6, wherein displaying said selectable option relating to an operational setting comprises displaying a selectable setting to adjust at least one of a current amplitude, a voltage amplitude, a rate of change of said current amplitude, a rate of change of said voltage amplitude, a time period of a rate of change of said current amplitude, a time period of a rate of change of said voltage amplitude, a pulse width, a rate of change of the pulse width, a time period of a rate of change of the pulse width, a frequency, a rate of change of the frequency, a time period of a rate of change of the frequency, a signal on-time, a signal off-time, and a duty cycle.

8. The method of claim 6, wherein guiding said medical procedure comprises prompting a health care provider to perform a plurality of steps relating to performing a surgical process for implanting an implantable medical device in a portion of a patient's body.

9. The method of claim 6, guiding said medical procedure comprises prompting a health care provider to perform a plurality of steps relating to a follow-up evaluation of an operation of said implantable medical device.

10. A method for guiding a medical procedure relating to operatively coupling an implantable medical device to a cranial nerve, comprising:
establishing communications between said implantable medical device and an external device;
transmitting a first signal from said external device to said implantable medical device;
receiving with said external device a second signal from said implantable medical device responsive to said first signal;
determining a recommendation using said external device, in response to said second signal; and
generating a display indicative of said recommendation using said external device.

11. The method of claim 10, wherein transmitting said first signal comprises providing a interrogative signal relating to a surgical implanting procedure for implanting said implantable medical device.

12. The method of claim 10, wherein transmitting said first signal comprises providing a interrogative signal relating to a system diagnostic process during a patient follow-up procedure.

13. The method of claim 10, further comprising:
performing a process responsive to said display indicative of said recommendation;
transmitting a third signal from said external device to said implantable medical device;
receiving with said external device a fourth signal from said implantable medical device responsive to said third signal;
determining a second recommendation using said external device, in response to said fourth signal; and
generating a second display indicative of said second recommendation using said external device.

14. The method of claim 13:
wherein transmitting said first signal comprises transmitting a signal to said implantable medical device to perform an first impedance test;
wherein receiving said second signal comprises receiving a signal indicating an a first high impedance result associated with a lead set coupled with said implantable medical device;
wherein generating said display indicative of said recommendation using said external device comprises displaying a recommendation to perform a lead adjustment upon said lead set;
wherein transmitting a third signal comprises transmitting a signal to perform a second impedance test subsequent to performing said lead adjustment;
wherein receiving said fourth signal comprises receiving a signal indicating a second high impedance result associated with said lead set coupled with said implantable medical device; and
wherein generating said display indicative of said second recommendation using said external device comprises displaying a recommendation to disconnect said lead set from said implantable medical device and perform a termination resistor test upon said implantable medical device.

15. A graphical user interface integrated into an external display device for guiding an operation relating to an implantable medical device coupled to a cranial nerve, comprising a display region adapted to display a visual indication of a graphical representation confirming the establishment of communications between said implantable medical device and said external device,
wherein said external device 1) transmits a first signal to said implantable medical device; 2) receives a second signal from said implantable medical device responsive to said first signal, and 3) determines a recommendation in response to said second signal,
and wherein said display region also provides a display indicative of said recommendation.

16. The graphical user interface integrated into said display device of claim 15, wherein said first signal comprises an interrogative signal relating to a surgical implant procedure for implanting said implantable medical device.

17. The graphical user interface integrated into said display device of claim 15, wherein first signal comprises providing an interrogative signal relating to a system diagnostic process during a patient follow-up procedure.

18. The graphical user interface integrated into said display device of claim 15, wherein said external device is further capable of 4) transmitting a third signal to said implantable medical device in response to performing a process responsive to said display indicative of said instruction, 5) receiving a fourth signal from said implantable medical device responsive to said third signal, and 6) determining a second instruction for said implantable medical device in response to said fourth signal, and wherein said display region further provides a display indicative of said second instruction.

19. A system for guiding an operation of an implantable medical device operatively coupled to a cranial nerve, comprising:
an implantable medical device capable of providing an electrical signal for treating a disease; and
an external computing device capable of communicating with said implantable medical device, the external computing device being capable of 1) establishing communications with said implantable medical device; 2) transmitting a first signal from said external device to said implantable medical device; 3) receiving a second signal from said implantable medical device responsive to said first signal; 4) determining an recommendation in response to said second signal; and 5) generating a display indicative of said recommendation using said external device.

20. The system of claim 19, wherein said implantable medical device is capable for treating a disease relating to at least one of a neuropsychiatric disorder selected from the group consisting of depression, autism, attention deficit/hyperactivity disorder; epilepsy; an eating disorder selected from the group consisting of bulimia, compulsive overeacting, obesity, bulimia, and anorexia nervosa; traumatic brain injury; stroke; coma; migraine; neuropathic pain; a cardiac condition selected from the group consisting of ischemia, congestive heart failure, and angina; sleep disorders; and a dementia disorder.

21. The system of claim 19, wherein said external device is at least one of a hand-held computing device, a standalone desktop computer system, a network computer system, a personal digital assistant (PDA) a pen input device, an electronic wand, and a portable computer device.

22. The system of claim 19, wherein said external device comprises a graphical user interface to display said recommendation, said graphical user interface to receive an input from a user in response to displaying said recommendation.

23. The system of claim 22, wherein further comprising:
a database unit to store data for use by said external unit for determining said recommendation; and
a remote device communicatively coupled to said external device and synchronized with said graphical user interface, said remote device to display the contents displayed by said graphical user interface.
